(19) United States
(12) Patent Application Publication
(10) Pub. No.: US 2012/0220548 A1
(43) Pub. Date: Aug. 30, 2012

(54) Compositions and methods for treatment of virus-associated cancer cells

(75) Inventors: BRYAN P. TOOLE, MT. PLEASANT, SC (US); CHRISTOPHER H. PARSONS, CHARLESTON, SC (US)

(73) Assignee: MEDICAL UNIVERSITY OF SOUTH CAROLINA, CHARLESTON, SC (US)

(21) Appl. No.: 13/406,106

(22) Filed: Feb. 27, 2012

Related U.S. Application Data
(60) Provisional application No. 61/447,525, filed on Feb. 28, 2011.

(51) Int. Cl. A61K 31/728; A61P 35/00

(52) U.S. Cl. 514/61; 514/54

(57) ABSTRACT

Compositions, methods and kits are provided for treating a cancer, tumor or pre-cancerous tissue condition resistant to a chemotherapeutic agent, the tissue condition having one or more proteins or tumorigenesis markers induced by, upregulated by or otherwise associated with virus exposure. A marker may be a receptor for, or may operatively regulate production or use of hyuronan, for example by mediating a hyaluronan-associated signal path or affecting expression of a protein or signaling pathway of the diseased tissue. A treatment composition includes a competitor of hyaluronan interactions and further includes or is co-administered with a drug, e.g., a chemotherapy agent to which the virus-associated condition would be resistant absent the hyaluronan or competitor.

### RELATED APPLICATION

The present application claims the benefit of U.S. provisional application Ser. No. 61/447,525 entitled, “Compositions, methods and kits for treating a cancer associated with a virus” with inventors Bryan P. Toole and Christopher H. Parsons, filed in the U.S. Patent and Trademark Office Feb. 28, 2011, and which is hereby incorporated herein by reference in its entirety.

### GOVERNMENT FUNDING

The invention herein was supported in part by grants from the National Institutes of Health R01-CA142362, R01-CA073839 and R01-CA082867, and a grant from the Department of Defense OC050368. The government has certain rights in the invention.

### TECHNICAL FIELD

The present invention relates to tissue treatment, and particularly to treatment of a cancer or precancerous tissue condition associated with a virus including the development and characteristics of the tissue condition include a history of exposure to a virus, and lesions associated with such exposure, generally culminating in an aggressive, invasive localized tissue tumor.

### BACKGROUND

The association with a virus as a primary etiological agent, and the latency stage lesions, such as body cavity lesions not localized in a specific organ, suggest a developmental history in which the blood cell immune responses may have incorporated viral DNA fragments, giving rise to lines of irregular B cells that, if not controlled, initiate invasive growth processes and form the tumor.

Many specific cancer cell lines have been characterized as exhibiting one or more specific complement display (CD) molecules on their cell surface, potentially allowing the development of delivery vehicles that target those CD molecules to deliver cytotoxic agents to the cell surface. Moreover, in better-studied cancer lines, the complement display molecules may serve as a diagnostic ‘finger print’ or confirmation of the associated cancer cell line, and research has often determined the functional roles performed by these complement display molecules, providing useful information for clinical intervention. However, the functional pathology of a virus-associated tumor is not so clear, and the specific roles played by its characteristic surface molecules may be complex and largely unknown. Virus-associated cancers, occurring in immunocompromised hosts with a history of cytotoxic drug treatment, may be drug-resistant, a factor that complicates the problem of treatment and results in high mortality.

Primary Effusion Lymphoma (PEL) is a lymphoma associated with Kaposi's sarcoma and its causative agent, the Kaposi sarcoma associated herpes virus (KSHV) also called human herpes virus-8 (HHV-8). Cytotoxic chemotherapy represents the standard of care for PEL, but high mortality is associated with PEL, partly due to the resistance of these tumors to chemotherapy. The membrane-bound glycoprotein emmprin (CD147) occurs in PEL, and it has been identified, in other tumor contexts, as a membrane bound inducer of matrix metalloproteinase synthesis, and promoter of tumor growth and invasiveness, enhancing chemoresistance in tumors through effects on transporter expression, trafficking and interactions. Interactions between hyaluronan and hyaluronan receptors on the cell surface are also known to facilitate chemoresistance. However, whether emmprin or hyaluronan-receptor interactions regulate chemotherapeutic resistance for virus-associated malignancies such as PEL remains unknown.

It is therefore desirable to provide more effective treatment of virus-associated cancers and more effective treatment compositions and treatment regimens for such cancers. It is also desirable to determine cellular mechanisms or responses driving growth processes such as invasive vascularization and uncontrolled growth or immortality, so as to determine appropriate and effective treatments for PEL and virus-associated disorders.

### SUMMARY OF EMBODIMENTS OF THE INVENTION

These and other desirable results are achieved herein based on the discovery coupled effects and mechanisms of activity of surface-bound proteins found in virus-associated cancer cells, at least one of which is related to, utilizes or is targeted by hyaluronan, and at least one of which is operative in tumorigenisis: deregulation or disruption of cellular processes, development of drug resistance or processes promoting tissue adhesion, invasion and/or vascularization. The invention provides treatments to impede, interrupt or abrogate these disease mechanisms, or reduce expression of proteins that mediate the mechanisms, and may further include methods of diagnosis and monitoring. Treatment methods include modulating hyaluronan interactions or administering a competitor to modulate such interactions, and sensitizing the affected cells to a drug thereby treating the cancer. Embodiments of the invention are illustrated in detail herein for PEL, a lymphoma associated with Kaposi's sarcoma and HHV-8. The invention also includes treatments for Epstein-Barr related or other virus-related conditions, and may be advantageously applied to cancerous or unregulated tissue disease conditions arising from or associated with a chronic viral infection such as herpesvirus, papilloma, influenza, or other oncoviruses.

Using human PEL tumor cells, the inventors demonstrate herein that PEL sensitivity to chemotherapy is related to expression of emmprin, the lymphatic vessel endothelial hyaluronan receptor (LYVE-1) and a drug transporter known as the breast cancer resistance protein/ABCG2 (BCRP). We further demonstrate that emmprin, LYVE-1 and BCRP interact with each other and colocalize on the PEL cell surface. In addition, experimental results show that emmprin induces chemoresistance in PEL cells through upregulation of BCRP expression, and that RNA interference targeting of emmprin, LYVE-1 or BCRP enhances PEL cell apoptosis induced by chemotherapy. Finally, disruption of hyaluronan-receptor interactions using small hyaluronan oligosaccharides reduces expression of emmprin and BCRP while sensitizing PEL cells to chemotherapy. Collectively, these data establish interdependent roles for emmprin, LYVE-1 and BCRP in chemotherapeutic resistance for PEL, and establish the treatment value of administering a cytotoxic agent and small hyaluronan oligosaccharides to treat PEL tumor cells. In other virus-induced conditions the treatment may target or disrupt VEGF expression or Akt-dependent disease associated proteins.

### BRIEF DESCRIPTION OF THE DRAWINGS

FIG. 1 is a photograph and a set of bar graphs showing that chemoresistance of PEL cells correlates directly with LYVE-1, emmprin and BCRP expression.

FIG. 1 panel A is a photograph of immunoblot analyses used to detect basal expression of emmprin, LYVE-1, and BCRP for chemosensitive (BC-1 and BC-3) and chemoresistant (BCP-1 and BCBL-1) PEL lines of cells. (3-Actin was identified for internal controls. Data shown represent one of three independent experiments.

FIG. 1 panel B is a bar graph of data from flow cytometric analyses used to quantify emmprin, LYVE-1, and BCRP expression on the surface of representative chemosensitive (BC-3) and chemoresistant (BCP-1) PEL cells. Mean fluorescence intensity (MFI), reflecting surface

expression of each protein for 10,000 cells in each condition, was calculated for BCP-1 cells relative to BC-3 cells using Flow To software.

FIG. 1 panel C is a bar graph of hyaluronan secretion in culture supernatants quantified as described in Examples.

FIG. 1 panel D is a bar graph of quantity of transcripts representing the three hyaluronan synthase genes (has1-3) quantified by qRT-PCR, and their expression relative to that for BC-1 cells determined as described in Examples. Error bars represent the standard error of the mean (S.E.M.) for three independent experiments. ** indicates p less than 0.01; *indicates p less than 0.05.

FIG. 2 is a set of photographs showing that emmprin, LYVE-1, and BCRP interact on the PEL cell surface.

FIG. 2 panel A shows results from confocal immunofluorescence assay (IFA) performed as described in Examples to identify expression and localization of emmprin, LYVE-1 and BCRP for BCP-1 cells. Red and green fluorescence represent localization of a single protein, while yellow fluorescence represents co-localization of two proteins in merged images. Data shown represent one of three independent experiments and at least 100 cells analyzed for each experiment.

FIG. 2 panels B-C show co-immunoprecipitation (Co-IP) assays performed as described in Examples. Proteins were identified within total protein (input) fractions for positive controls, and IgG antibodies of the same subclass were used for negative controls for both anti-emmprin (panel B) and anti-LYVE-1 co-IP (panel C).

FIG. 3 is a set of photographs, bar graphs and line graphs showing that targeting emmprin reduces BCRP expression, hyaluronan secretion and PEL cell resistance to chemotherapeutic agents. BCP-1 cells were transfected with emmprin-specific siRNA (e-siRNA) or non-target control siRNA (n-siRNA). After 48 h, immunoblot analyses were used to quantify protein expression FIG. 3 panel A, supernatants used for quantification of hyaluronan secretion FIG. 3 panel B, and flow cytometric analyses used to quantify emmprin, BCRP and LYVE-1 expression on the cell surface FIG. 3 panel C. For the latter, MFI, reflecting surface expression of each protein for 10,000 cells, was determined for e-siRNA-treated BCP-1 cells relative to controls. FIG. 3 panel D shows confocal IFA performed to identify and localize emmprin and BCRP expression as described in Examples. FIG. 3 panel E shows cells e-siRNA-transfected or n-siRNA control-transfected cells (24 h) incubated with the indicated concentrations of paclitaxel (Taxol) or doxorubicin (Dox) for 72 h and relative cell viability quantified using trypan blue exclusion as described in Examples. For all experiments, error bars represent the S.E.M. for three independent experiments. ** indicates p less than 0.01.

FIG. 4 is a set of photographs and line graphs showing that emmprin induces PEL resistance to chemotherapy through induction of BCRP expression.

FIG. 4 panel A shows a western blot analysis of BC-1 cells transduced using a recombinant human emmprin-encoding adenovirus (AdV-emmprin), or control adenovirus (AdV), and protein expression quantified 48 h later by immunoblotting.

FIG. 4 panel B shows viability of BC-1 cells transfected with control non-target- (n) or BCRP (b)-specific siRNA for 24 h, then transduced as in (A) for an additional 48 h prior to incubation with the indicated concentrations (nM on x-axes) of Taxol (left panel) or Dox (right panel) for 72 h each. Relative cell viability was quantified using trypan blue exclusion. Error bars represent the S.E.M. for three independent experiments.

FIG. 4 panel C shows viability of BCBL-1 cells transfected with BCRP-siRNA or non-target control siRNA (n-siRNA) for 48 h, then immunoblot analyses used to detect BCRP expression.

FIG. 4 panel D shows viability of cells following transfection as in (C), of BCBL-1 cells that were incubated with Taxol or Dox for 72 h at the indicated concentrations and relative cell viability quantified using trypan blue exclusion.

FIG. 5 is a bar graph and a set of line graphs showing that emmprin induces PEL resistance to chemotherapy through induction of hyaluronan-receptor interaction.

FIG. 5 panel A shows data from BC-1 cells transduced as in FIG. 4 and supernatants used for quantification of hyaluronan secretion after 48 h.

FIG. 5 panel B shows data from BC-1 cells transduced as in panel A for 48 h, then incubated with either Taxol or Dox at the indicated concentrations, in the presence or absence of 100 μg/mL oHA, for 72 h. Relative cell viability was quantified using trypan blue exclusion. Error bars represent the S.E.M. for three independent experiments.

FIG. 6 is a set of photographs, a bar graph, and line graphs showing that targeting LYVE-1 reduces BCRP expression and PEL cell resistance to chemotherapeutic agents. BCP-1 cells were transfected with LYVE-1-siRNA or non-target control siRNA (n-siRNA). After 48 h, immunoblot analyses were used to quantify protein expression FIG. 6 panel A and flow cytometric assays used to quantify LYVE-1 and BCRP expression on the cell surface FIG. 6 panel B. For the latter, MFI, reflecting surface expression of each protein for 10,000 cells, was determined for LYVE-1-siRNA-treated BCP-1 cells relative to controls. FIG. 6 panel C shows data from confocal IFA used to identify and localize LYVE-1 and BCRP expression as described in Examples. FIG. 6 panel D shows data from LYVE-1-siRNA-transfected or n-siRNA control-transfected BCP-1 cells incubated with Taxol or Dox for 72 h at the indicated concentrations, and cell viability quantified using trypan blue exclusion. Error bars represent the S.E.M. for three independent experiments. ** indicates p less than 0.01.

FIG. 7 is a set of cell flow cytometry data and a bar graph that shows that targeting emmprin or LYVE-1 enhances PEL cell apoptosis induced by chemotherapeutic agents.

FIG. 7 panel A shows BCP-1 cells transfected with emmprin-siRNA (e-siRNA), LYVE-1-siRNA (1-siRNA) or non-target control siRNA (n-siRNA) for 24 h, then incubated in the presence or absence of 100 nM Dox for an additional 24 h. Apoptosis was quantified by flow cytometry using Annexin V and PI as described in Examples.

FIG. 7 panel B shows the percentage of total (early+late) apoptotic cells within at least 10,000 cells in each group per experiment that was determined as described in Examples. Error bars represent the S.E.M. for three independent experiments. ** indicates p less than 0.01.

FIG. 8 is a set of line graphs, flow cytometry data, and a photograph showing that oHA sensitize chemoresistant PEL cells to chemotherapeutic agents. BCP-1 (FIG. 8 panels A-B) and BCBL-1 cells (FIG. 8 panels C-D) were incubated with either Taxol or Dox at the indicated concentrations and for the indicated times in the presence or absence of 100 μg/mL oHA. Relative cell viability was quantified using trypan blue exclusion. Error bars represent the S.E.M. for three independent experiments. FIG. 8 panel E shows data from BCP-1 cells that were incubated with 100 nM Taxol or 100 nM Dox in the presence or absence of 100 μg/mL oHA for 48 h, then apoptosis quantified by flow cytometry as described in Examples. FIG. 8 panel F shows immunoblots of cells treated as in FIG. 8 panel E, to identify apoptosis-associated protein expression as described in Examples. Data shown for FIG. 8 panels E and F represent one of three independent experiments. FIG. 9 is a bar graph and a set of photographs showing that oHA reduce emmprin and BCRP expression in PEL cells treated with chemotherapeutic agents.

FIG. 9 panel A shows BCP-1 cells were incubated with 100 nM Taxol or 100 nM Dox for 96 h in the presence or absence of 100 μg/mL oHA. Immunoblot analyses were used to detect total protein expression, including β-Actin for internal controls. Data shown represent one of three independent experiments.

FIG. 9 panel B shows flow cytometry analyses were used to quantify BCRP cell surface expression for similar conditions as in (A). MFI, reflecting surface expression of BCRP for 10,000 cells, was determined for experimental groups relative to untreated BCP-1 control cells. Error bars represent the S.E.M. for three independent experiments *indicates p less than 0.05; ** indicates p less than 0.01.

FIG. 9 panel C shows BCP-1 cells treated as in (A), then confocal IFA performed for identification and localization of BCRP expression as described in Examples. Data shown represent one of three independent experiments.

FIG. 10 is a set of photographs showing that PEL cells incubated with oHA exhibit increased intracellular accumulation of doxorubicin. BCP-1 cells were incubated with 100 nM Dox for 48 h in the presence or absence of 100 μg/mL oHA. then confocal IFA were performed to identify intracellular doxorubicin (green) as described in Examples. For identification of nuclei (blue), cells were counterstained with 0.5 μg/mL 4′,6-diamidino-2-phenylindole (DAPI; Sigma) in 180 mM Tris-HCl (pH 7.5), and visualization of nuclear fragmentation was used to identify cells undergoing apoptosis (arrows). Data shown represent one of three independent experiments. See Qin Z, et al. Leukemia 2011; 25: 1598-1609, which is incorporated by reference herein in its entirety, for all purposes including visualization of colors.

FIG. 11 is a set of photographs showing immunoblots of chemoresistant PEL cells, and shows that those cells exhibit greater expression of emmprin-associated metallomatrix proteinases (MMPs). Immunoblot analyses were used to detect basal expression of MMP 1, MMP2 and MMP9 for both chemosensitive (BC-1 and BC-3) and chemoresistant (BCP-1 and BCBL-1) PEL cells. β-actin was identified for internal controls. Data shown represent one of three independent experiments.

FIG. 12 is a set of bar graphs showing that oHA alone do not induce PEL cytotoxicity. BC-1 FIG. 12 panel A, BC-3 FIG. 12 panel B, BCP-1 FIG. 12 panel C and BCBL-1 FIG. 12 panel D were incubated with the indicated concentrations of oHA for 96 h and cell viability was determined using a standard MTT assay according to the manufacturer's instructions and confirmed by trypan blue exclusion. Error bars represent the S.E.M. for three independent experiments.

FIG. 13 is a set of line graphs that show that oHA enhance cytotoxicity for chemosensitive PEL cells in the presence of chemotherapeutic agents. BC-1, FIG. 13 panels A-B and BC-3, FIG. 13 panels C-D were incubated with either Taxol or Dox at the indicated concentrations and for the indicated times in the presence (squares) or absence (diamonds) of 100 μg/mL oHA. Relative cell viability was quantified using trypan blue exclusion as described in Examples. Error bars represent the S.E.M. for three independent experiments.

FIG. 14 is a set of photographs of immunoblots showing that oHA alone do not affect expression of emmprin, LYVE-1, or BCRP in PEL cells. BCP-1 and BCBL-1 cells were incubated in the presence or absence as indicated of 100 μg/mL oHA for 96 h, then immunoblot analyses were used to detect total protein expression, including β-actin for internal controls. Data shown represent one of three independent experiments.

FIG. 15 is a set of photographs of immunoblots showing that oHA reduce interaction of emmprin and BCRP with LYVE-1 in PEL cells treated with chemotherapeutic agents. BCP-1 cells were incubated with 100 nM Taxol or 100 nM Dox for 48 h in the presence or absence of 100 μg/mL oHA as indicated. Co-IP assays were then performed as described in Examples.

FIG. 16 is a line graph showing effect of oHA in combination with rapamycin on relative cell viability of drug-resistant primary effusion lymphoma (PEL) cells in culture, on the ordinate, as a function of concentration of rapamycin, nM, on the abscissa, on a log scale. The cells used are the PEL strain known as body cavity-based lymphoma-1 (BCBL-l; diamonds and squares). The squares indicate data from cells to which oHA was added along with rapamycin. The data show that oHA sensitized the cells to killing by rapamycin by at least about twenty-fold, as about 50% survival was observed at 1 nM of rapamycin in the presence of oHA, compared to absence of oHA for rapamycin.

FIG. 17 is a line graph showing effect of oHA in combination with rapamycin on growth of tumors in BCBL-1-injected NOD/SCID mice. Mice were injected with 2×10⁷ BCBL-1 cells and were weighed as a function of time every other day for one month, to assess tumor growth. The data show that rapamycin (administered intraperitoneally at a dose of 0.2 mg/kg) in combination with oHA (administered intraperitoneally at a dose of 0.5 mg/kg; data shown as -x-) substantially reduced progress of lymphoma, as mouse weight was similar to that of control mice not injected with BCBL-1 cells (diamonds). In contrast, mice administered rapamycin alone (triangles) or control mice administered vehicle (squares) developed substantial tumor-associated weight gain (about 4 g, representing a weight gain of more than 15%). The weight is shown on the ordinate and time in days on the abscissa.

FIG. 18 is a line graph showing effect of oHA in combination with doxorubicin on growth of tumors in BCBL-1-injected NOD/SCID mice. Mice were injected with 2×10⁷ BCBL-1 cells and were weighed as a function of time every week for 3 weeks, to assess tumor growth. The data show that doxorubicin (administered intraperitoneally at a dose of 0.2 mg/kg) in combination with oHA (administered intraperitoneally at a dose of 0.5 mg/kg; data shown as—black line/circles) substantially reduced progress of lymphoma, as mouse weight was only slightly greater than that of control mice not injected with BCBL-1 cells (blue line/diamonds). In contrast, mice administered doxorubicin alone (green line/triangles) or control mice administered vehicle (red line/squares) developed substantial tumor-associated weight gain (4-7g, representing a weight gain of approximately 35% from baseline weight of 25-28g). The weight is shown on the ordinate and time in days on the abscissa.

FIG. 19 is a set of photographs of western blot data showing upregulation of protein expression following primary human endothelial cell (EC) infection with KSHV, or EC transfection by the KSHV-encoded protein: LANA. EC extracts analyzed in the panel on the left were transformed with a vector encoding LANA (pc-LANA) or a control vector (pc), and expression of BCRP was analyzed and shown to be upregulated by LANA. EC extracts in the right panel show that LANA also upregulates expression of CD44 and LYVE-1, as does KHSV infection in comparison to uninfected EC (mock). Actin expression was used as a loading control and was not affected by any of these treatments.

FIG. 20 is a set of photographs of western blot data showing relative amounts of activated Akt (p-Akt) and activated mTOR (p-mTOR) in BCBL1 Doxorubicin treated cells with and without oHA, with β-actin as the control loading. No differences were observed in the total levels of Akt or mTOR, but the levels of activated Akt and activated mTOR, important signaling pathways in tumorigenesis, were substantially reduced in the oHA-treated cells.

### DETAILED DESCRIPTION

The Kaposi's sarcoma-associated herpesvirus (KSHV) is the etiologic agent of primary effusion lymphoma (PEL; Cesarman E, et al. N Engl J Med 1995; 332(18): 1186-1191), multi-centric Castleman's disease (Soulier J, et al. Blood 1995; 86(4): 1276-1280) and Kaposi's sarcoma (Chang Y, et al. Science 1994; 266(5192): 1865-1869). PEL represents a rapidly progressive illness arising primarily in patients infected with the human immunodeficiency virus (HIV), although cases have also been documented in organ transplant recipients. Administration of cytotoxic chemotherapeutic agents represents the current standard of care for the treatment of PEL (Simonelli C, et al. J Clin Oncol 2003; 21(21): 3948-3954; Boulanger E, et al. J Clin Oncol 2005; 23(19): 4372-4380; Chen Y B, et al. Oncologist 2007; 12(5): 569-576. However, the myelosuppressive effects of cytotoxic chemotherapy synergize with those caused by antiretroviral therapy or immune suppression (Petre C E, et al. J Virol 2007; 81(4): 1912-1922; Munoz-Fontela C, et al. J Virol 2008; 82(3): 1518-1525).

Furthermore, the prognosis for PEL remains poor with a median survival of approximately six months, dictating the need for safer and more effective therapeutic options. Therapies targeting the mammalian target of rapamycin (mTOR or CD20) have proven helpful in select cases (Oksenhendler E, et al. Am J Hematol 1998; 57(3): 266; Hocqueloux L, et al. AIDS 2001; 15(2): 280-282), although a lack of efficacy due to induction of alternative tumor-promoting signal transduction pathways or outgrowth of CD20-negative tumors limits the utility of these approaches. Many PEL tumors demonstrate resistance to chemotherapeutic agents used in the clinic. p53 mutagenesis and the KSHV-encoded latency-associated nuclear antigen-2 (LANA2) have been implicated in PEL resistance to chemotherapy, but a better understanding of mechanisms for PEL chemoresistance is needed in order to develop clinically applicable approaches for sensitizing PEL tumors to cytotoxic agents.

Emmprin (CD147; basigin) was originally identified as a membrane-bound inducer of matrix metalloproteinase (MMP) synthesis (Biswas C, et al. Cancer Res 1995; 55(2): 434-439; Guo H, et al. J Biol Chem 1997; 272(1): 24-27), enhanced tumor growth, and tumor cell invasion (Zucker S, et al. Am J Pathol 2001; 158(6): 1921-1928). More recent studies have demonstrated emmprin interactions with monocarboxylate and ATP-binding cassette (ABC)-family multidrug transporters to facilitate export of lactate or chemotherapeutic agents, respectively (Kirk P, et al. EMBO J2000; 19(15): 3896-3904; Gallagher S M, et al. Cancer Res 2007; 67(9): 4182-4189; Gallagher S M, et al. Cancer Res 2007; 67(9): 4182-4189; Slomiany M G, et al. Cancer Res 2009; 69(4): 1293-1301; Slomiany M G, et al. Clin Cancer Res 2009; 15(24): 7593-7601; Wang W J, et al. Chemotherapy 2008; 54(4): 291-301).

Emmprin also stimulates production of hyaluronan (Marieb E A, et al. Cancer Res 2004; 64(4): 1229-1232), an extracellular polysaccharide that promotes tumor chemoresistance through interactions with the cell surface receptor CD44 (Slomiany M G, et al. Cancer Res 2009; 69(12): 4992-4998; Gilg, A. G., et al. Clin Cancer Res. 14:1804-1813, 2008; Misra S, et al. J Biol Chem 2003; 278(28): 25285-25288; Misra S, et al. J Biol Chem 2005; 280(21): 20310-20315Torre C, et al. Arch Otolaryngol Head Neck Surg 2010; 136(5): 493-501). Small hyaluronan oligosaccharides (oHAs) interact monovalently with CD44, competitively blocking polyvalent interactions between CD44 and endogenous hyaluronan (Lesley J, et al. J Biol Chem 2000; 275(35): 26967-26975; Underhill C B, et al. J Biol Chem 1983; 258(13): 8086-8091), and oHAs sensitize murine lymphoma, malignant peripheral nerve sheath tumor, glioma and various carcinoma cell lines to chemotherapy in vitro and in vivo (Slomiany M G, et al. Clin Cancer Res 2009; 15(24): 7593-7601; Slomiany M G, et al. Cancer Res 2009; 69(12): 4992-4998; Gilg, A. G., et al. Clin Cancer Res. 14:1804-1813, 2008; Misra S, et al. J Biol Chem 2003; 278(28): 25285-25288; Misra S, et al. J Biol Chem 2005; 280(21):

20310-20315; Cordo Russo R I, et al. Int J Cancer 2008; 122(5): 1012-1018). The lymphatic vessel endothelial hyaluronan receptor-1 (LYVE-1), which has structural similarity to CD44, also serves as a receptor for hyaluronan (Jackson D G. Immunol Rev 2009; 230(1): 216-231). Interestingly, LYVE-1 is expressed by KSHV-infected cells and within KSHV- associated tumors (Carroll P A, et al. Virology 2004; 328(1): 7-18; An F Q, et al. J Virol 2006; 80(10): 4833-4846; Pyakurel P, et al. Int J Cancer 2006; 119(6): 1262-1267), although a role for LYVE-1 in KSHV pathogenesis has not been established. Furthermore, surface expression of CD44 is negligible for PEL cells (Boshoff C, et al. Blood 1998; 91(5): 1671-1679). It is unknown whether emmprin, hyaluronan receptors or other associated proteins regulate chemotherapeutic resistance for virus-mediated tumors.

Using patient-derived PEL tumors, applicants determined that PEL cells express emmprin, LYVE-1 and the ABC family transporter known as the breast cancer resistance protein/ABCG2 (BCRP) on the cell surface. Therefore, we sought to determine whether emmprin, LYVE-1 and/or BCRP, either alone or through interdependent interactions, regulate PEL resistance to chemotherapeutic agents.

Applicants have discovered that the proliferation of diseased cells or growth of tumors could be effectively addressed by providing a competitor of hyaluronan interactions to and/or silencing expression of a disease-related protein to increase apoptosis of diseased cells and/or sensitize resistant cells to a treatment agent. The competitor of hyaluronan interactions may be a small hyaluronan oligomer (o-HA) which competes with hyaluronan, a decoy that competitively binds to hyaluronan, or may include DNA or RNA adapted to reduce expression of or to inactivate an associated marker or protein. In an embodiment, the oligomer reduces resistance to the drug or agent, and the agent reduces viability of the cancer or tumor, thereby treating the treating the cancer or tissue condition. Methods are illustrated below to treat a primary effusion lymphoma associated with the human herpes virus HHV-8 and Kaposi's sarcoma. The small oligomers (oHAs) may have a molecular size distribution under about twenty disaccharides in length, and preferably between about three and twelve disaccharides in length. A suitable RNA intervention includes siRNA that negatively modulates nucleic acid encoding a virus-associated surface marker, which may for example be selected from the group of: emmprin, breast cancer resistance protein (BCRP), and lymphatic vessel endothelial hyaluronic acid receptor (LYVE-1). Other tumorigenisis markers may include VEGF, CD44 or other proteins associated with viral infection by Epstein-Barr virus (EBV), human papilloma virus (HPV), HIV, cytomegalovirus or other virus that is chronic or persistent in an immuno-compromised host. Compositions and treatment methods of the invention are useful in overcoming drug resistance, a common treatment problem that arises because patients afflicted with such viral agents often undergo multiple courses of antiviral, antibacterial or anticancer chemotherapy. The resistant cells of a virus-associated precancerous tissue condition may comprise highly differentiated cells (for example, having drug resistant B-cells as the principal etiologic agent) that become particularly invasive or aggressive when contacting certain tissue types, and the treatment compositions of the present invention may be seen as causing affected cells to de-differentiate, restoring susceptibility to drug treatment or disrupting their diseased or mis-regulated cellular processes.

HA is a high molecular weight glycosaminoglycan (GAG) that is distributed ubiquitously in vertebrate tissues, and is expressed at elevated levels in many tumor types. In breast cancer cells, the level of hyaluronan concentration is a negative predictor of survival. HA-tumor cell interactions are shown herein to lead to enhanced activity of the phosphoinositide-3-kinase/Akt cell survival pathway and that small hyaluronan oligosaccharides antagonize endogenous hyaluronan polymer interactions, stimulating phosphatase and tensin (PTEN) expression and suppressing the cell survival pathway. Under anchorage-independent conditions, HA oligomers (oHA) inhibit growth and induce apoptosis in cancer cells.

The chemotherapeutic drugs used herein represent three classes of chemicals that are commonly used for cancer patients and to which tumors are resistant. Resistance to apoptosis in monolayer culture and in spheroid culture, where resistance is often enhanced, is tested. Finally, resistance of tumors in vivo to treatment with chemotherapeutic agents in the presence of HA oligomers is tested in nude mice xenografts to ensure that results obtained in culture apply in vivo.

Multi-drug resistance of cancer cells remains a serious problem in treatment today. Since HA oligomers are non-toxic and non-immunogenic, they may provide a novel avenue for improving the efficacy of chemotherapy in cancer patients. HA oligomers are shown herein to retard tumor growth in vivo. The possibility that these oligomers also reverse chemoresistance by increasing cell susceptibility to chemotherapeutic agents may lead to novel treatments that enhance current chemotherapeutic protocols.

Increased amounts of hyaluronan are shown herein to enhance tumor cell survival and suppress tumor cell death, thus promoting tumor growth and metastasis. Shorter lengths of an HA polymer (HA “oligomers”) antagonize the effect of full-size, polymeric HA. HA oligomers have now been found to act by suppressing biochemical reactions that may be important in promoting multi-drug resistance to chemotherapy.

HA is a linear glycosaminoglycan composed of 2,000-25,000 disaccharides of glucuronic acid and N-acetylglucosamine: [β1,4-GlcUA-β1,3-GlcNAc-]ₙ, with molecular weights ranging from 10⁵ to 10⁷ daltons (Da). The disaccharide subunit has a molecular weight of 400 Da. Hyaluronan synthases (termed Has1, Has2, Has3) are integral plasma membrane proteins whose active sites are located at the intracellular face of the membrane (Weigel, P et al. 1997; J Biol Chem 272: 13997-14000). Newly synthesized HA is extruded directly onto the cell surface; it is either retained there by sustained attachment to the synthase or by interactions with receptors, or it is released into pericellular and extracellular matrices. Regulation of targeting to these various locations is not understood at this time.

HA has multiple physiological and cellular roles that arise from its unique biophysical and interactive properties (reviewed in Toole, B. P., et al. Cell Dev Biol, 12: 79-87, 2001; Toole, B. P., et al. Glycobiology, 12: 37R-42R, 2002). There are at least three ways in which HA can influence normal and abnormal cell behavior. First, due to its biophysical properties, free HA has a profound effect on the biomechanical properties of extracellular and pericellular matrices in which cells reside. Second, hyaluronan forms a repetitive template for specific interactions with other pericellular macromolecules, thus contributing to the assembly, structural integrity and physiological properties of these matrices. Thus, HA makes extracellular matrix more conducive to cell shape changes required for cell division and motility (Hall, C. L., et al. J Cell Biol, 126: 575-588, 1994; Evanko, S. P., et al. Arterioseler Thromb Vase Biol, 19: 1004-1013, 1999). Third, H A interacts with cell surface receptors that transduce intracellular signals and influence cellular form and behavior directly (Turley, E et al. 2002; J Biol Chem 277: 4589-4592).

### Therapeutically Effective Dose

In yet another aspect, according to the methods of treatment of the present invention, the treatment of a virus-associated cancer is promoted by contacting the cancer cells with a pharmaceutical composition, as described herein. Thus, the invention provides methods for the treatment of tumors comprising administering a therapeutically effective amount of a pharmaceutical composition comprising active agents that include oHA to a subject in need thereof, in such amounts and for such time as is necessary to achieve the desired result. It will be appreciated that this encompasses administering an inventive pharmaceutical as a therapeutic measure to promote the sensitization of the virus-associated cancer cells or a virus-associated tumor to a chosen therapeutic agent, particularly a chemotherapeutic agent.

In certain embodiments of the present invention a “therapeutically effective amount” of the pharmaceutical composition is that amount effective for promoting killing of the cancer cell, for example, inducing apoptosis of a cancer cell in the presence of the therapeutic agent. The compositions, according to the method of the present invention, may be administered using any amount and any route of administration effective for increased loss of cancer cell viability. Thus, the expression “amount effective to overcome invasiveness, drug resistance or metastasis characteristics of the cell or tumor, or to induce cell death for a virus-infected cell or tumor ” as used herein, refers to a sufficient amount of composition to reduce or eliminate growth and/or size of the tumor or cancer. The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., tumor size and location; age, weight and gender of the patient; diet, time and frequency of administration; drug combinations; reaction sensitivities; and tolerance/response to therapy. Long acting pharmaceutical compositions might be administered every three to four days, every week, or once every two weeks depending on half-life and clearance rate of the particular composition. Pharmaceutical compositions can be compounded that contain both oHA and the anti-cancer chemotherapeutic drug, or the oHA and chemotherapeutic drug can be compounded separately.

The active agents of the invention are preferably formulated in dosage unit form for ease of administration and uniformity of dosage. The expression “dosage unit form” as used herein refers to a physically discrete unit of active agent appropriate for the patient to be treated. It will be understood, however, that the total daily usage of the compositions of the present invention will be decided by the attending physician within the scope of sound medical judgment. For any active agent, the therapeutically effective dose can be estimated initially either in cell culture assays or in animal models as shown in examples herein, usually mice, rabbits, dogs, or pigs. The animal model is also used to achieve a desirable concentration range effective for the co-administering active anti-cancer agent, and route of administration. Such information can then be used to determine useful doses and routes for administration in humans. A therapeutically effective dose refers to that amount of active agent which ameliorates the symptoms or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use.

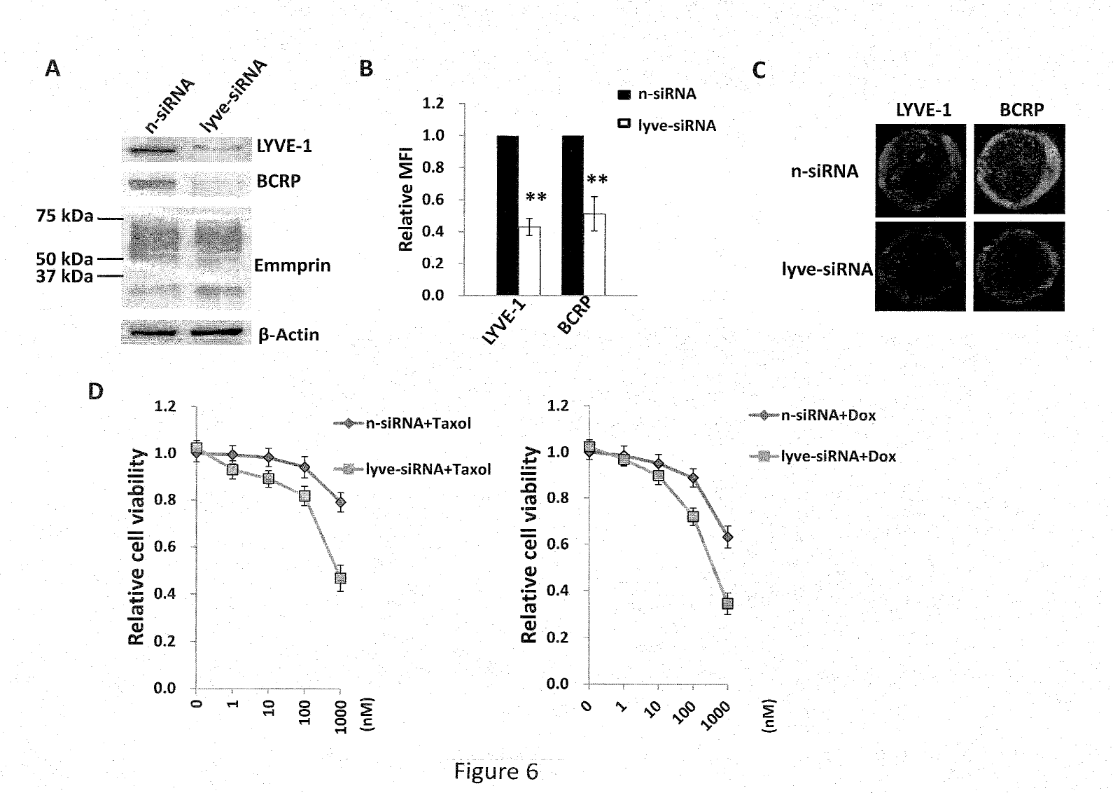

Data in examples herein show that 0.5 mg/kg oHA is sufficient to get a maximum effect when combined with a chemotherapeutic agent—see attachment 1, FIG. 6, panel C. Further, a dose as great as 250 mg/kg have been used without observations of signs of toxicity (FIG. 6, attachment 1, panel A), for systemic delivery. A lower dose is effective for intratumoral or for topical administration to an epithelial tumor.

Accordingly, the compositions of the present invention include a systemic or intratumoral dose from about 0.1 mg/kg to about 0.2 mg/kg, from about 0.2 mg/kg to about 0.5 mg/kg, from about 0.4 mg/kg to about 0.6 mg/kg, from about 0.1 mg/kg to about 1.0 mg/kg, from about 0.1 mg/kg to about 2 mg/kg, from about 0.2 mg/kg to about 20 mg/kg, and from about 0.1 mg/kg to about 50 mg/kg.

### Administration of Pharmaceutical Compositions

After formulation with an appropriate pharmaceutically acceptable carrier in a desired dosage, the pharmaceutical compositions of this invention can be administered to humans and other mammals topically (as by powders, ointments, or drops), orally, rectally, parenterally, intracistemally, intravaginally, intraperitoneally, bucally, ocularly, or nasally, depending on the severity and location of the tumor being treated.

Liquid dosage forms for oral administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of the inventive oHA pharmaceutical composition to superficial tumors include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, ocular or cutaneous tumors may be treated with aqueous drops, a. mist, an emulsion, or a cream. Administration may be therapeutic or it may be prophylactic. Prophylactic formulations may be present or applied to the site of potential tumors, or to sources of tumors, such as contact lenses, contact lens cleaning and rinsing solutions, containers for contact lens storage or transport, devices for contact lens handling, eye drops, surgical irrigation solutions, ear drops, eye patches, and cosmetics for the eye area, including creams, lotions, mascara, eyeliner, and eyeshadow. The invention includes ophthalmological devices, surgical devices, audiological devices or products which contain disclosed compositions (e.g., gauze bandages or strips), and methods of making or using such devices or products. These devices may be coated with, impregnated with, bonded to or otherwise treated with a disclosed composition.

The ointments, pastes, creams, and gels may contain, in addition to an active agent of this invention, excipients such as animal and vegetable fats, oils, waxes, paraffins, starch, tragacanth, cellulose derivatives, polyethylene glycols, silicones, bentonites, silicic acid, talc, zinc oxide, or mixtures thereof.

Powders and sprays can contain, in addition to the agents of this invention, excipients such as talc, silicic acid, aluminum hydroxide, calcium silicates, polyamide powder, or mixtures of these substances. Sprays can additionally contain customary propellants such as chlorofluorohydrocarbons.

Transdermal patches have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations for systemic administration or for intratumoral injection, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection.

Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Compositions for rectal or vaginal administration for treatment of epithelial tumors in these locations are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage fauns may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract for treatment of tumors or polyps, optionally, in a delayed manner. Examples of embedding compositions which can be used include polymeric substances and waxes.

### Uses of Pharmaceutical Compositions

As discussed above and described in greater detail in the Examples, oHA compositions are shown herein to be useful as sensitizers of tumors to well characterized anti-cancer therapeutic agents, and accordingly it is envisioned to additional chemotherapeutic agents as these are discovered. In general, it is believed that oHAs will be clinically useful in promoting apoptosis of cancer cells resulting from virus contact, for example, viruses of the Herpes and papilloma family, and retroviruses, including in lymphomas of hematopoietic origin, and in tumors associated with any epithelial and endothelial tissue, including but not limited to the skin epithelium; the corneal epithelium; the lining of the gastrointestinal tract; the lung epithelium; and the inner surface of kidney tubules, of blood vessels, of the uterus, of the vagina, of the urethra, or of the respiratory tract; and to endothelial tumors and tumors arising from non-epithelial cells. These cancers may be identified in normal individuals or in subjects having conditions which result in reduced immune surveillance of potential transformed cells, such as virus exposure, and such exposure alone or in combination with diabetes, corneal dystrophies, uremia, malnutrition, vitamin deficiencies, obesity, infection, immunosuppression and complications associated with systemic treatment with steroids, radiation therapy, non-steroidal anti-inflammatory drugs (N SAID), anti-neoplastic drugs and anti-metabolites.

In general, the oHA compositions herein are useful as sensitizing agents, to be administered in conjunction with a standard therapeutic regimen, and will be found to reduce amounts or frequencies of dosages of that regimen. Whether compounded together or separately, the oHA and drug can be administered together or separately, using the same, similar or different administration regimens.

It will be appreciated that the therapeutic methods encompassed by the present invention are not limited to treating tumors in humans, but may be used to treat tumors in any mammal including but not limited to bovine, canine, feline, caprine, ovine, porcine, murine, and equine species, for example high value agricultural, zoo and sports animals.

### EXAMPLES

Experimental investigations and the resulting discoveries are set forth below. The following materials and methods were used throughout subsequent examples.

### Example 1

### Cell Culture

KSHV-infected PEL cells, including BC-1, BC-3, BCP-1 and BCBL-1 cell lines, were provided by the laboratories of Dr. Dean H. Kedes (University of Virginia) and Dr. Dirk Dittmer (University of North Carolina, Chapel Hill). All PEL cells were maintained in RPMI-1640 media (Gibco, Gaithersburg, Md., USA) supplemented with 10% fetal bovine serum, 10 mM HEPES (pH 7.5), 100U/ml penicillin, 100 μg/ml streptomycin, 2 mM L-glutamine, 0.05 mM β-mercaptoethanol and 0.02% (wt/vol) sodium bicarbonate.

### Example 2

### Preparation of Hyaluronan Oligomers (oHAs)

oHAs were prepared as described in Slomiany M G, et al. Cancer Res 2009; 69(4): 1293-1301. Briefly, the oHA preparation comprises a mixed fraction of average molecular weight (MW) ˜2.5×10³ composed of 3 to 10 disaccharide units fractionated from testicular hyaluronidase (type 1-S) digests of hyaluronan polymer (Sigma-Aldrich (St Louis, Mo., USA), sodium salt). Fractionation was performed using trichloroacetic acid precipitation followed by serial dialysis with 5000 MWCO (Amicon Ultra Ultracel, Millipore, Billerica, Mass., USA) and 1000 MWCO (Spectra/Por Membrane, Spectrum Laboratories, Rancho Dominguez, Calif., USA) membranes.

### Example 3

### Cell Viability Assays

Cell viability was assessed using both MTT and Trypan blue exclusion assays as described in Qin Z, et al. PLoS Pathog 2010; 6(1): e1000742. For MTT assays, a total of 5×10³ PEL cells were incubated individual wells of a 96-well plate for 24 hours. Serial dilutions of paclitaxel, doxorubicin or oHAs were added and subsequently incubated in 1 mg/ml MTT solution (Sigma-Aldrich) at 37° C. for 3 hours. Thereafter, cells were incubated in 50% dimethylsulfoxide overnight and optical densities determined at 570 nm using a spectrophotometer (Thermo Labsystems, West Palm Beach, Fla., USA). For Trypan blue exclusion assays, cells were incubated with 0.4% Trypan blue (MP Biomedicals, Northbrook, Ill., USA) and observed under light microscopy. Relative cell viability was determined after assessment of at least 1000 cells per condition for each experiment using the following formula: (no. of live cells/no. of total cells for experimental conditions)/ (no. of live cells/no. of total cells for vehicle-treated control cells).

### Example 4

### Gene Amplification

Total RNA was isolated using the RNeasy Mini kit according to the manufacturer's instructions (QIAGEN, Valencia, Calif., USA). Complementary DNA was synthesized from equivalent concentrations of total RNA using the SuperScript III First-Strand Synthesis SuperMix Kit (Invitrogen, Carlsbad, Calif., USA) according to the manufacturer's instructions. Coding sequences for hyaluronan synthases 1-3 (has1-3) and β-actin for internal controls were amplified from 200 ng input complementary DNA using iQ SYBR Green Supermix (Bio-Rad, Hercules, Calif., USA). Custom primer sequences used for amplification experiments were as follows:

| (SEQ ID NO: 1) | |
|---|---|
| has1 sense | 5′-CAAGGCGCTCGGAG ATTC-3′; |
| (SEQ ID NO: 2) | |
| has1 antisense | 5′-GACCGCTGATGCAGGATACA-3′; |
| (SEQ ID NO: 3) | |
| has2 sense | 5′-CATCATCCAAAGCCTGTT-3′; |
| (SEQ ID NO: 4) | |
| has2 antisense | 5′-TCTTCTGAGTTCCCATCTA-3′; |
| (SEQ ID NO: 5) | |
| has3 sense | 5′-TGGCTCAACC AGCAAACC-3′; |
| (SEQ ID NO: 6) | |
| has3 antisense | 5′-CAGCAGGAAGAGGAGA ATGT-3′; |
| (SEQ ID NO: 7) | |
| β-actin sense | 5′-GGAAATCGTGCGTGACATT-3′; |
| and, | |
| (SEQ ID NO: 8) | |
| β-actin antisense | 5′-GACTCGTCATACTCCTGCTTG-3′. |

Amplification was carried out using an iCycler IQ Real-Time PCR Detection System, and cycle threshold (Ct) values determined in duplicate for emmprin has transcripts and β-actin for each experiment. ‘No template’ (water) and ‘no-RT’ controls were used to ensure minimal background DNA contamination. Fold changes for experimental groups relative to assigned controls were calculated using automated iQ5 2.0 software (Bio-Rad).

### Example 5

### RNA Interference (RNAi)

Emmprin, LYVE-1, BCRP and non-target small interfering RNAs were purchased from the manufacturer (ON-TARGET plus SMART pool, Dharmacon, Lafayette, Colo., USA). Cells were incubated with small interfering RNAs in 12-well plates using DharmaFECT Transfection Reagent (Dharmacon) according to the manufacturer's instructions, and gene silencing assessed using immunoblots within 48 hours.

### Example 6

### Immunoprecipitation and Immunoblot Assays

Cells were lysed in buffer containing 20 mM Tris (pH 7.5), 150 mM NaCl, 1% NP40, 1 mM EDTA, 5 mM NaF and 5 mM Na₃VO₄. Total cell lysates (30 μg) were resolved by 10% sodium dodecyl sulfate polyacrylamide gel electrophoresis, transferred to nitrocellulose membranes, and immunoblotted with 100-200 μg/ml antibodies recognizing the following proteins: BCRP, LYVE-1 (Santa Cruz, Santa Cruz, Calif., USA), Bax, pro-/cleaved caspase-9, pro-/cleaved caspase-3, Bel-2 (Cell Signaling, Boston, Mass., USA) and emmprin (BD Pharmingen, San Jose, Calif., USA). For loading controls, blots were reacted with antibodies detecting β-actin (Sigma-Aldrich). Immunoreactive bands were developed using an enhanced chemiluminescence reaction (Perkin-Elmer, San Jose, Calif., USA), and visualized by autoradiography. Immunoprecipitation assays were performed using the Catch and Release v2.0 Reversible Immunoprecipitation System (Millipore) according to the manufacturer's instructions (Invitrogen). Mouse or rabbit IgG were used as negative controls.

### Example 7

### Flow Cytometry

PEL cells were resuspended in 3% bovine serum albumin in lx phosphate-buffered saline, incubated on ice for 10 min, and then incubated with primary antibodies (diluted 1:50 for emmprin, and 1:20 for BCRP and LYVE-1) for an additional 30 min. Following two subsequent wash steps, cells were incubated for an additional 30 min with either goat anti-rabbit IgG Alexa-647 or goat anti-mouse IgG Alexa-647 (Invitrogen) diluted 1:200. Control cells were incubated with secondary antibodies only. Cells were resuspended in 1×phosphate-buffered saline before analysis. For quantitative apoptosis assays, the fluorescein isothiocyanate Annexin V Apoptosis Detection Kit I (BD Pharmingen) and propidium iodide were used according to the manufacturer's instructions to identify early apoptotic (annexin⁺propidium iodide) and late apoptotic (annexin⁺propidium iodide⁺) cells for 10000 cells in each experimental and control condition. Data were collected using a FACS Calibur four-color flow cytometer (Bio-Rad), and FlowJo software (TreeStar, San Carlos, Calif., USA) was used to quantify cell surface localization of target proteins. The percentage of total apoptotic cells in each sample was calculated as follows: (early apoptotic+late apoptotic cells)/total cells analyzed.

### Example 8

### Immunofluorescence Assays

PEL cells were incubated in 3% paraformaldehyde at 4° C. for fixation, and then with a blocking reagent (3% bovine serum albumin in lx phosphate-buffered saline) for an additional 30 min. Cells were subsequently incubated for 1 hour at 25° C. with primary antibodies (diluted 1:50 for emmprin, and 1:20 for BCRP and LYVE-1), followed by goat anti-rabbit IgG Texas Red or goat anti-mouse IgG Alexa-488 (Invitrogen) diluted 1:100 for an additional 1 h at 25° C. To detect the presence of doxorubicin within individual cells, doxorubicin was excited using an argon laser (λₑₓ=488 nm) and detected using an emission filter set at 505-530 nm, as described by Mellor et al, 2011. Images were captured using a Leica TCS SP5 AOBS confocal microscope (Leica Microsystems Inc., Buffalo Grove, Ill., USA) equipped with a X63/1.4 objective lens.

### Example 9

### Transduction Assays

PEL cells were transduced (multiplicity of infection approximately 20) using a recombinant adenoviral vector encoding emmprin or a control vector as previously described (Li R, et al. J Cell Physiol 2001; 186(3): 371-379). After 24 hours, cells were incubated with paclitaxel and doxorubicin (Sigma-Aldrich) with or without 100 μg/ml oHA before quantification of cell viability.

### Example 10

### Hyaluronan Quantification

Hyaluronan concentrations were determined in cell supernatants using an enzyme-linked immunosorbent-like assay accordingly to Gordon L B, et al. Hum Genet 2003; 113(2): 178-187.

### Example 11

### Statistical Analysis

Significance for differences between experimental and control groups was determined using the two-tailed Student's t-test (Excel 8.0), and P-values less than 0.05 or less than 0.01 were considered significant or highly significant, respectively.

### Example 12

### Chemoresistance of PEL Cells to Correlate Directly with LYVE-1, Emmprin and BCRP Expression

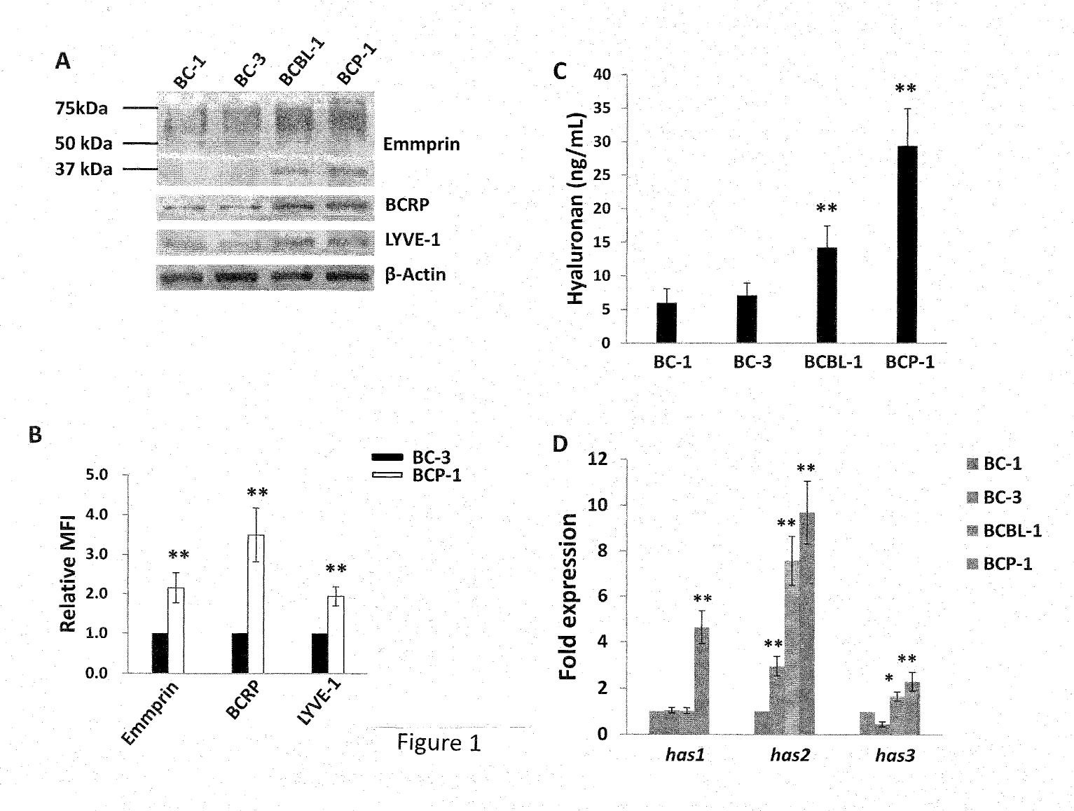

As shown in FIG. 1 panel A, immunoblot analyses were used to detect basal expression of emmprin, LYVE-1 and BCRP for relatively chemosensitive PEL cells (BC-1 and BC-3) and for chemoresistant PEL cells (BCP-1 and BCBL-1). β-actin was identified for internal controls. Data shown in Panel A represent one of three independent experiments. Panel B shows flow cytometric analyses used to quantify emmprin, LYVE-1 and BCRP expression on the surface of representative chemosensitive (BC-3) and chemoresistant (BCP-1) PEL cells. Mean fluorescence intensity (MFI), reflecting surface expression of each protein for 10 000 cells in each condition, was calculated for BCP-1 cells relative to BC-3 cells using FlowJo software. Hyaluronan secretion in culture supernatants was quantified as described in the Materials and methods section supra, and is shown in panel C. In addition, transcripts representing three hyaluronan synthase genes (has1-3) were quantified by qRT-PCR, and their expression relative to that for BC-1 cells determined as described supra; measurements are shown in Panel D; the error bars represent the s.e.m. for three independent experiments. * indicates P less than 0.05; ** indicates P less than 0.01.

### Example 13

### Emmprin, LYVE-1 and BCRP were Found to Interact on the PEL Cell Surface

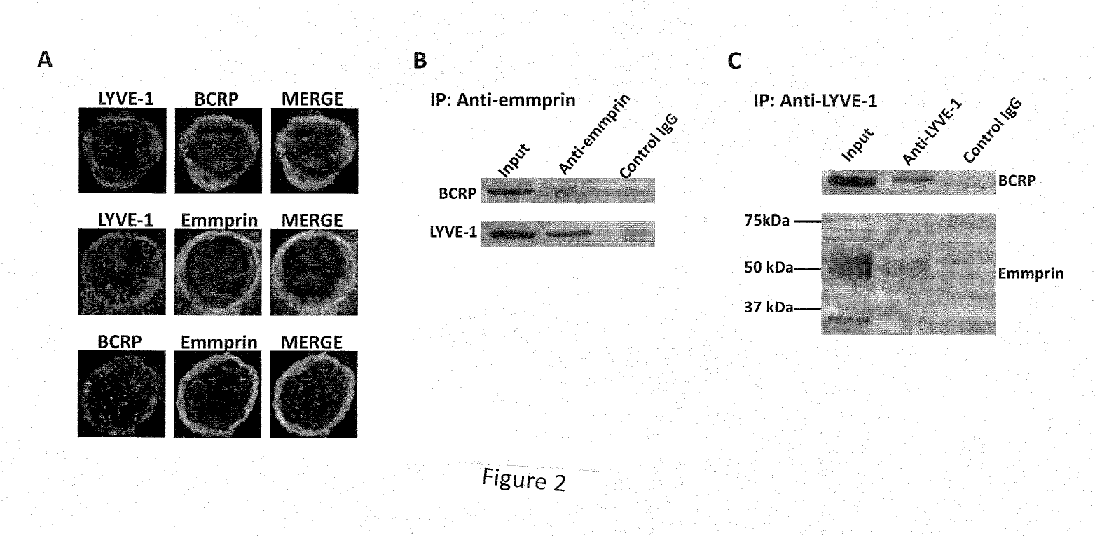

Confocal immunofluorescence assays (IFAS) were performed as described in the materials and methods examples supra, and were used to identify expression and localization of emmprin, LYVE-1 and BCRP using BCP-1 cells. Observing the original color images from which FIG. 2 panel A herein was prepared, red or green fluorescence represents localization of a single protein, whereas yellow fluorescence represents colocalization of two proteins in merged images. Data shown represent one of three independent experiments and at least 100 cells analyzed for each experiment. The color images are found in applicants now-published article, Qin Z et al., Leukemia 2011; 25: 1598-1609, which is hereby incorporated by reference herein in its entirety for all purposes. Panels B and C illustrate co-immunoprecipitation (co-IP) assays which were performed as described in the materials and methods examples supra. Proteins were identified within total protein (input) fractions for positive controls, and IgG antibodies of the same subclass were used for negative controls for both anti-emmprin and anti-LYVE-1 co-IP assays.

Using four representative human PEL cell lines, we sought to determine whether chemoresistance for PEL cells correlates with their expression of emmprin, LYVE-1 and BCRP. We chose to focus on BCRP as we observed its clear expression on the PEL cell surface (FIG. 1 panel B), whereas we did not observe appreciable PEL cell expression of the other ubiquitous, well-characterized ABC transporter, P-glycoprotein. The present example used four human PEL cell lines: two chemoresistant cell lines (BCP-1 and BCBL-1 cells) and two chemosensitive cell lines (BC-1 and BC-3 cells), previously characterized based on their relative sensitivity to the DNA synthesis inhibitor doxorubicin (Petre C E, et al. J Virol 2007; 81(4): 1912-1922).

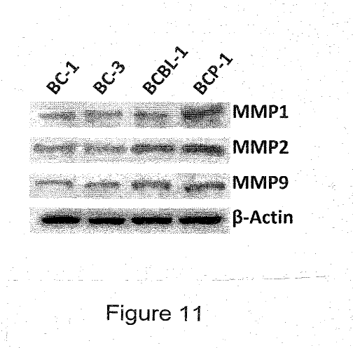

Using immunoblotting and flow cytometry, respectively, total protein expression and membrane localization of emmprin, LYVE-1 and BCRP were found to be significantly greater for chemoresistant PEL cells (FIGS. 1, panels A and B). Surprsingly, chemoresistant PEL cells exhibited greater expression of both high-MW (about 65kDa) and low-MW (about 35 kDa) emmprin glycoforms. Emmprin isoforms with high or low levels of glycosylation demonstrate biologic activity with respect to induction of MMP expression (Tang W, et al. Mol Biol Cell 2004; 15: 4043-4050; Belton Jr R J, et al. J Biol Chem 2009; 283: 17805-17811). Correlating with these results, greater expression of representative MMPs (MMPI, MMP2 and MMP9) in chemoresistant PEL cells was observed (FIG. 11). In addition, chemoresistant PEL cells exhibited increased hyaluronan secretion and greater expression of hyaluronan synthase transcripts (has1-3 for BCP-1; has⅔ for BCBL-1) relative to chemosensitive PEL cells (FIGS. 1, panels C and D). Protein complexes containing emmprin or CD44 and drug transporters have been previously identified on the surface of tumor cells (Slomiany M G, et al. Clin Cancer Res 2009; 15(24): 7593-7601; Slomiany M G, et al. Cancer Res 2009; 69(12): 4992-4998).

Examples herein using confocal microscopy showed colocalization of emmprin, LYVE-1 and BCRP on the PEL cell surface (FIG. 2, panel A). Moreover, BCRP and LYVE-1 co-immuno-precipitated with emmprin, and BCRP and emmprin co-immunoprecipitated with LYVE-1 (FIG. 2 panels B and C). These results support interactions between emmprin, LYVE-1 and BCRP on the surface of chemoresistant PEL cells. Further examples were therefore planned and performed to elucidate these coupled effects or interactions.

### Example 14

### Targeting Emmprin Reduces BCRP Expression, Hyaluronan Secretion and PEL Cell Resistance to Chemotherapeutic Agents

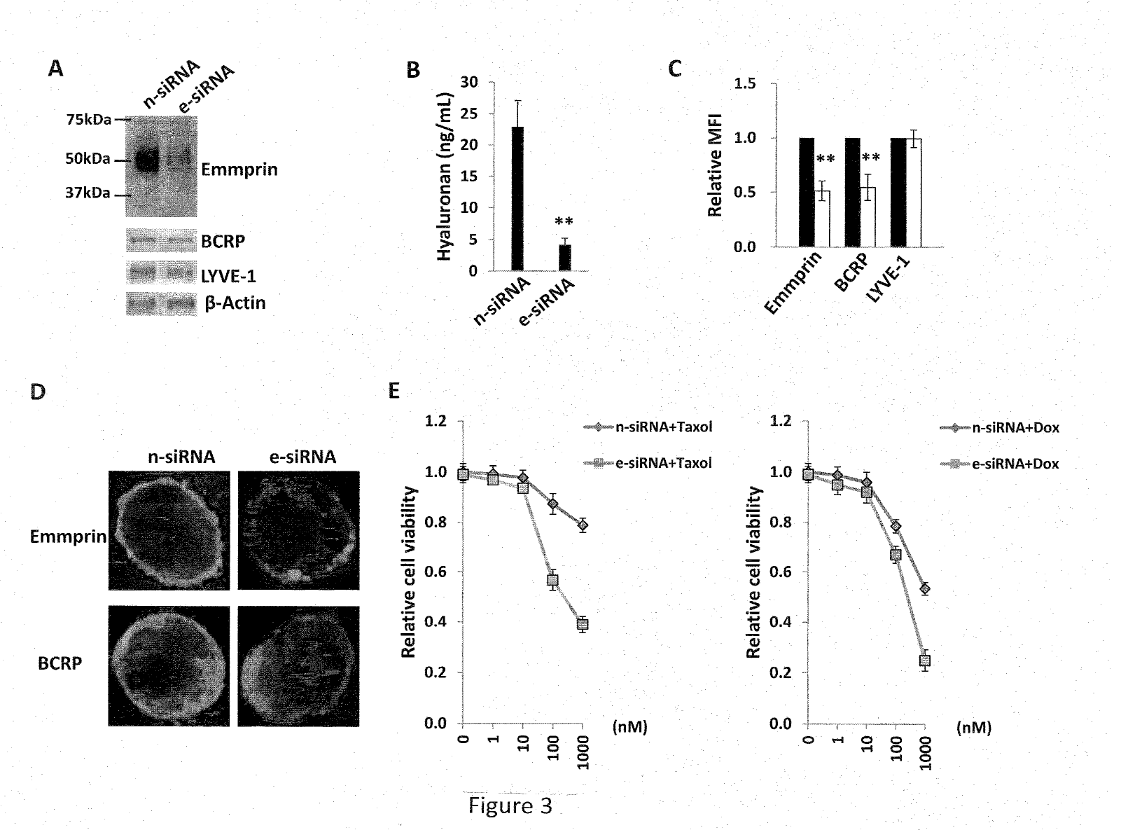

BCP-1 cells were transfected with emmprin-specific small interfering RNA (e-siRNA) or non-target control siRNA (n-siRNA). After 48 hours, immunoblot analyses were used to quantify protein expression (shown in FIG. 3, panel A). Supernatants were used to quantify hyaluronan secretion (shown in FIG. 3 panel B), and flow cytometric analyses were performed to quantify emmprin, BCRP and LYVE-1 expression on the cell surface (results shown in FIG. 3 panel C). For the latter, mean fluorescence intensities representing cell surface expression (MFI), following analysis of 10⁴ cells, were determined for e-siRNA-treated BCP-1 cells (white bars) relative to controls (black bars). Confocal IFAs were performed to identify and localize emmprin and BCRP expression as described in the examples with materials and methods, supra, and representative cell images showing emmprin and BCRP on the cell surface are shown in FIG. 3 panel D. In addition, e-siRNA-transfected or n-siRNA control-transfected cells were incubated for 24 hours with varying concentrations of paclitaxel (Taxol) or for 72 hours with doxorubicin (Dox) as shown in FIG. 3 panel E and relative cell viability was quantified using Trypan blue exclusion as described in the examples showing the materials and methods, supra. For all experiments, error bars represent the s.e.m. for three independent experiments. ** indicates P less than 0.01.

It was observed that following RNAi resulting in partial inhibition of emmprin expression in PEL cells, immunoblots (FIG. 3 panel A) show partial reduction of total BCRP protein expression, and no clearly discernible reduction in LYVE-1 expression. Inhibition of emmprin expression significantly reduced hyaluronan secretion by chemoresistant PEL cells (FIG. 3 panel B). Furthermore, flow cytometry and confocal microscopy demonstrated that inhibition of emmprin significantly reduced BCRP localization on the cell surface, but not LYVE-1 (FIG. 3 panels C and D). Doxorubicin is used routinely for the treatment of PEL (Chen Y B, et al. Oncologist 2007; 12(5): 569-576). The microtubule inhibitor paclitaxel also induces apoptosis of human PEL tumors in vitro (ang Y F, et al. Cancer Chemother Pharinacol 2004; 54(4): 322-330) but paclitaxel is not routinely used for the treatment of PEL due, in part, to the demonstration of PEL resistance to paclitaxel (Munoz-Fontela C, et al. J Virol 2008; 82(3): 1518-1525). The viability assays showed that targeting emmprin increased the sensitivity of chemoresistant PEL cells to both doxorubicin and paclitaxel (FIG. 3 panel E). Data herein showing sensitization of PEL cells to several chemotherapeutic anti-cancer agents by administering a modulator of hyaluronan receptors indicates that these agents can be successfully used to treat the virus-related cancers.

### Example 15

### Emmprin and LYVE-1 Regulate BCRP Expression and PEL Resistance to Chemotherapy

Further examples were performed to determine whether emmprin induces PEL resistance to chemotherapy through induction of BCRP expression. BC-1 cells were transduced using a recombinant human emmprin-encoding adenovirus (AdV-emmprin) or control adenovirus (AdV), and protein expression was quantified 48 hours later by immunoblotting.

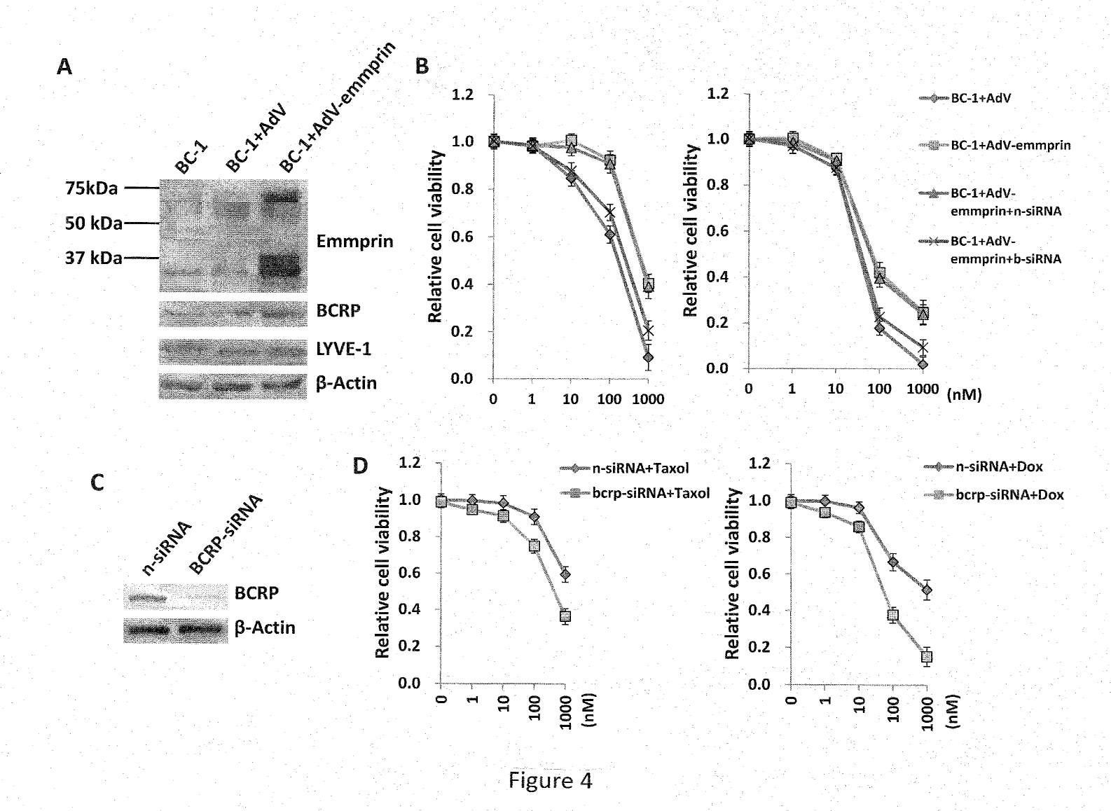

As shown in FIG. 4 panel A, ectopic overexpression of emmprin increased BCRP expression in chemosensitive PEL cells whereas LYVE-1 remained unaffected. Furthermore, emmprin overexpression significantly reduced PEL cell sensitivity to both doxorubicin and paclitaxel and, using RNAi, it was confirmed that this effect was mediated almost entirely through upregulation of BCRP (FIG. 4 panel C). BC-1 cells were transfected with control non-target- (n-) or BCRP-specific (brcp-) small interfering RNA (siRNA) for 24 hours, and then transduced as in Panel A for an additional 48 h before incubation with the indicated concentrations (nM on x axis) of Taxol (left panel) or Dox (right panel) for 72 h each. Relative cell viability was quantified using Trypan blue exclusion. Error bars represent the s.e.m. for three independent experiments. For Panel C, BCBL-1 cells were transfected with BCRP-siRNA or non-target control siRNA (n-siRNA) for 48 h, and then immunoblot analyses were used to detect BCRP expression. Following transfection as in (C), BCBL-1 cells were incubated with Taxol or Dox for 72 h at the indicated concentrations and relative cell viability quantified using Trypan blue exclusion, as shown in Panel D of FIG. 4.

Thus, using transduction with a recombinant adenovirus encoding emmprin, the data showed found (FIG. 4 panels A, B), and confirmed that using RNAi (FIG. 4 panels C, D) for reducing BCRP expression significantly enhanced PEL cytotoxicity induced by either doxorubicin or paclitaxel. These data show that the mechanism for enhancing toxicity of chemotherapeutic anti-cancer agents in otherwise resistant cells having a virus-associated cancer involves reducing BCRP expression.

### Example 16

### Chemoprotection by Emmprin Depends on Hyaluronon Receptor Interactions

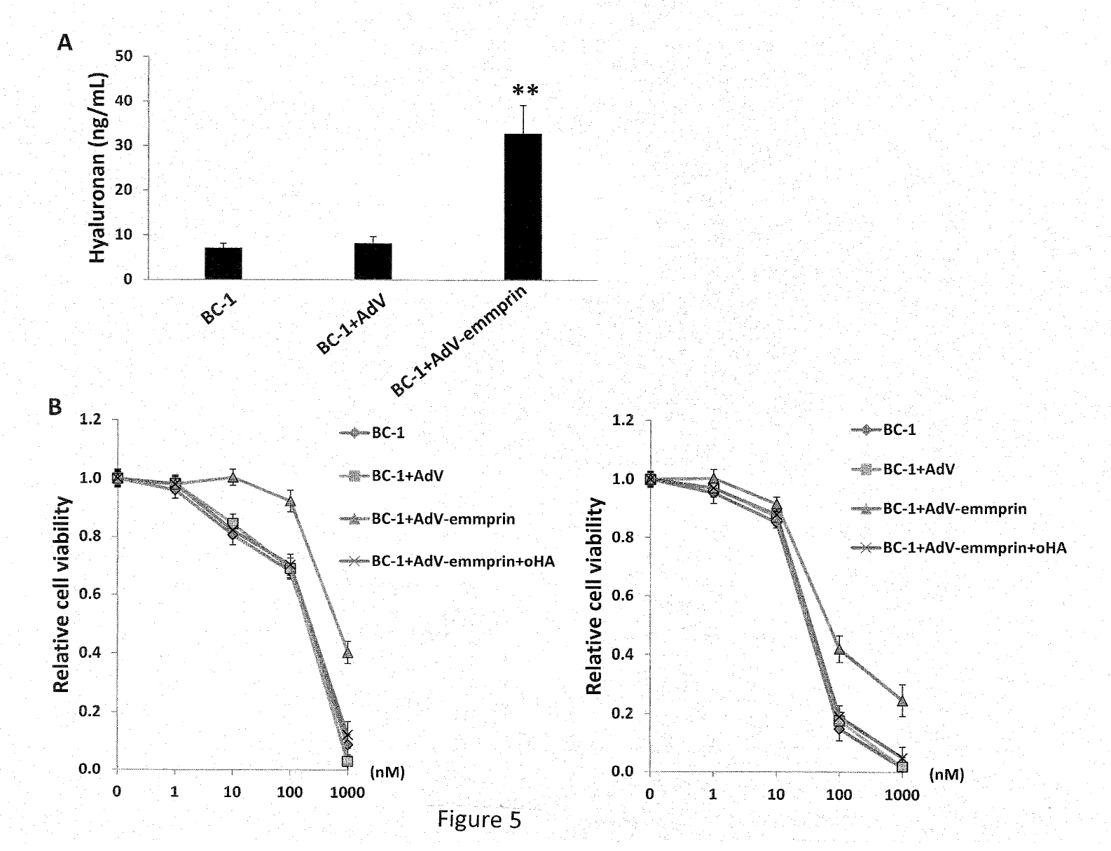

In this experiment, BC-1 cells were transduced as in FIG. 4 to induce emmprin overexpression, and supernatants were analyzed for quantification of hyaluronan secretion after 48 hours (FIG. 5 panel A), which shows an increase of about three-fold of hyaluronan secretion as a result of transduction with the gene encoding emmprin. Emmprin overexpression was observed to be significantly associated with increased hyaluronan secretion.

To assess effects on sensitization to chemotherapeutic drags, BC-1 cells were transduced as above for 48 hours and subsequently incubated with either Taxol (FIG. 5 panel B, left) or Dox (FIG. 5 panel B, right) at the indicated concentrations of the drugs, and in the presence or absence of 100 μg/ml oHA for an additional 72 hours. Relative cell viability was quantified using Trypan blue exclusion. Error bars represent the s.e.m. for three independent experiments.

It was observed from these data that the increase in chemoresistance caused by emmprin overexpression was effectively suppressed by co-administration of oHAs, indicating that the chemoprotective effect of emmprin for PEL cells is dependent upon hyaluronan-receptor interactions.

### Example 17

### Targeting LYVE-1 Reduces BCRP Expression and Lowers PEL Chemoresistance

Having observed LYVE-1 expression on the surface of PEL cells as well as oHA suppression of emmprin-mediated chemoresistance, it was envisioned that inhibition of LYVE-1 expression also would sensitize PEL cells to chemotherapy. It was observed in this example that RNAi targeting LYVE-1 reduced both total expression and membrane localization of BCRP in PEL cells, but did not affect emmprin expression significantly. Moreover, reduced LYVE-1 expression significantly enhanced PEL cell sensitivity to both doxorubicin and paclitaxel.

For this example, BCP-1 cells were transfected with LYVE-1-siRNA or with a non-target control small interfering RNA (n-siRNA). After 48 hours, immunoblot analyses were performed to quantify protein expression of LYVE-1, BCRP and Emmprin (shown in FIG. 6 panel A) and flow cytometric assays were used to quantify LYVE-1 and BCRP expression on the cell surface (FIG. 6 panel B). In FIG. 6 panel B, mean fluorescence intensities representing cell surface expression (MFI), following analysis of 10⁴ cells, were determined for LYVE-1-siRNA-treated BCP-1 cells (white bars) relative to controls (black bars). In addition, confocal immunofluorescence assays (IFAs) were used to identify and localize LYVE-1 and BCRP expression on the cells as described in the Materials and methods examples supra, and these images are shown in FIG. 6 panel C.

Drug sensitivity was assessed as follows: LYVE-1-siRNA-transfected or n-siRNA control-transfected BCP-1 cells were incubated with Taxol (FIG. 6 panel D, left graph) or Dox (FIG. 6 panel D, right graph) for 72 hours at the indicated drug concentrations, and cell viability was quantified using Trypan blue exclusion. Error bars represent the s.e.m. for three independent experiments. ** indicates P less than 0.01.

The data show that for each drug, LYVE-1-siRNA-transfected cells were rendered more chemosensitive than n-siRNA control transfected cells. These data show that targeting LYVE-1 reduced BCRP expression and lowered PEL cell resistance to chemotherapeutic agents, and did not significantly affect either type or amount of emmprin expression.

### Example 18

### PEL Chemoresistance is Regulated by Cooperative Mechanisms Involving Emmprin and Hyaluronan Interactions Affecting Apoptosis

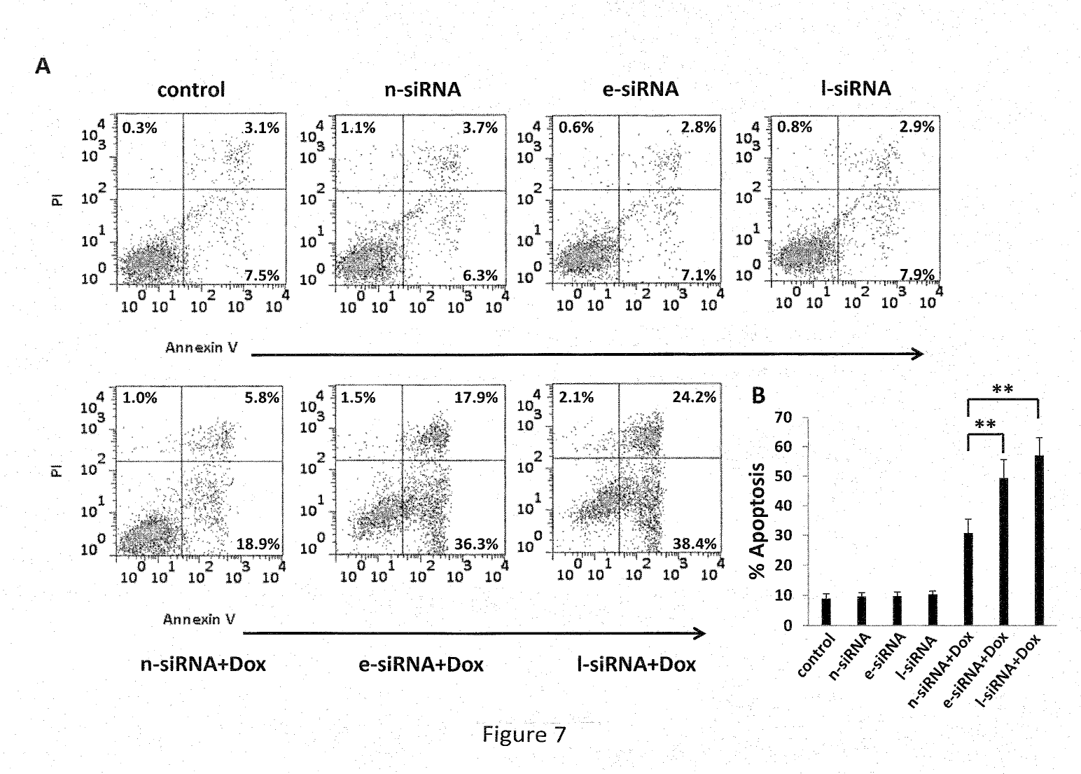

BCP-1 cells were transfected with emmprin-small interfering RNA (e-siRNA), LYVE-1-siRNA (1-siRNA) or non-target control siRNA (n-siRNA) for 24 hours, and then incubated in the presence or absence of 100 nM Dox for an additional 24 hours. Apoptosis was quantified by flow cytometry using Annexin V and propidium iodide and the data for these groups is shown in FIG. 7 panel A. The percentage of total (early plus late) apoptotic cells within at least 10⁴ cells in each group per experiment was determined as described in the examples containing materials and methods, supra, and these are illustrated in FIG. 7 panel B. Error bars represent the S.E.M. for three independent experiments, and ** indicates P less than 0.01.

The complimentary flow cytometric assays demonstrated that reduction in expression of either emmprin or LYVE-1 led to enhanced apoptosis in the presence of chemotherapeutic agents. However, no significant effect was observed when either emmprin or LYVE-1 was targeted in the absence of chemotherapeutic agent.

Collectively, these results indicate that cooperative mechanisms involving emmprin and hyaluronan interactions with LYVE-1 regulate PEL chemoresistance, and that upregulation of BCRP is responsible for these effects.

### Example 19

### oHA Enhances Amount of Apoptosis Induced by Chemotherapeutic Agents

Published data indicate that oHAs induce apoptosis for a lymphoma cell line (Cordo Russo R I., et al. Int J Cancer 2008; 122(5): 1012-1018; Alaniz L, et al. Glycobiology 2006; 16(5): 359-367). As shown in examples supra, oHAs suppress emmprin-induced chemoresistance for PEL cells (FIG. 5 panel B). Accordingly, further examples herein sought to explore whether oHAs reduce PEL viability through induction of apoptosis, and whether oHAs alone sensitize PEL cells to chemotherapic effects of anti-cancer drugs.

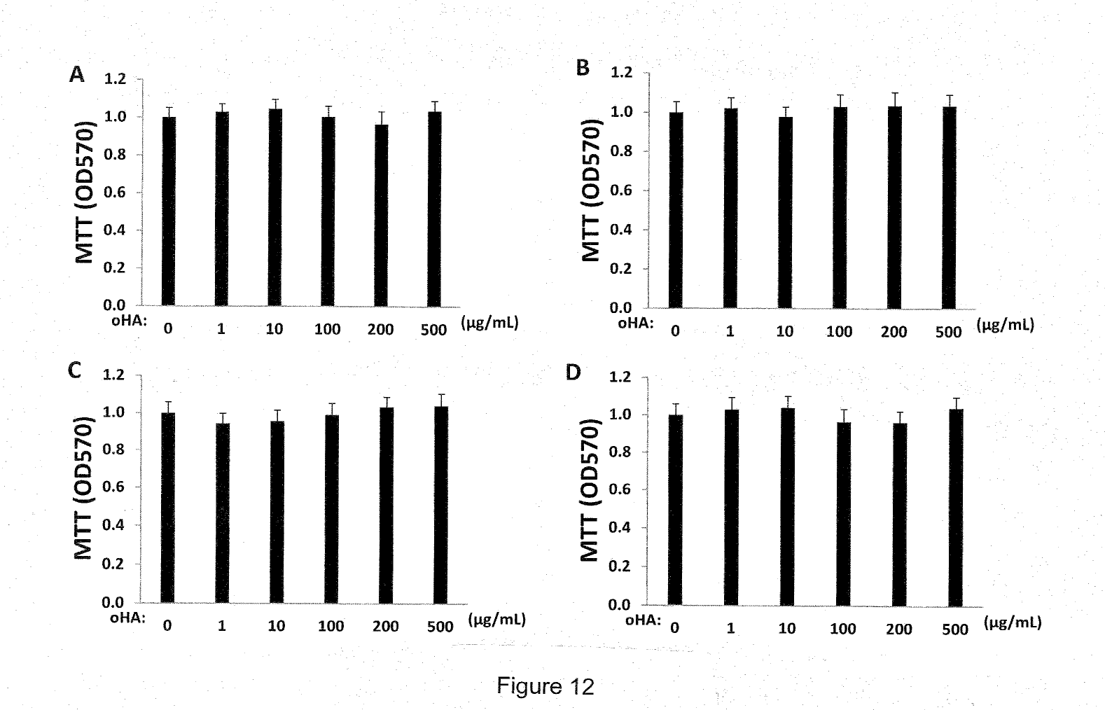

In agreement with our results herein indicating that RNAi targeting emmprin or LYVE-1 alone has no impact on PEL viability, it was observed that oHAs alone did not induce cytotoxicity for PEL cells. FIG. 12 panels A, B, C, and D show the results of a standard MTT viability assay according to the manufacturer's instructions for BC-1, BC-3, BCP-1 and BCBL-1 cells, a conlusion from which is that oHA alone does not induce PEL cytotoxicity. Error bars represent the s.e.m. for three independent experiments.

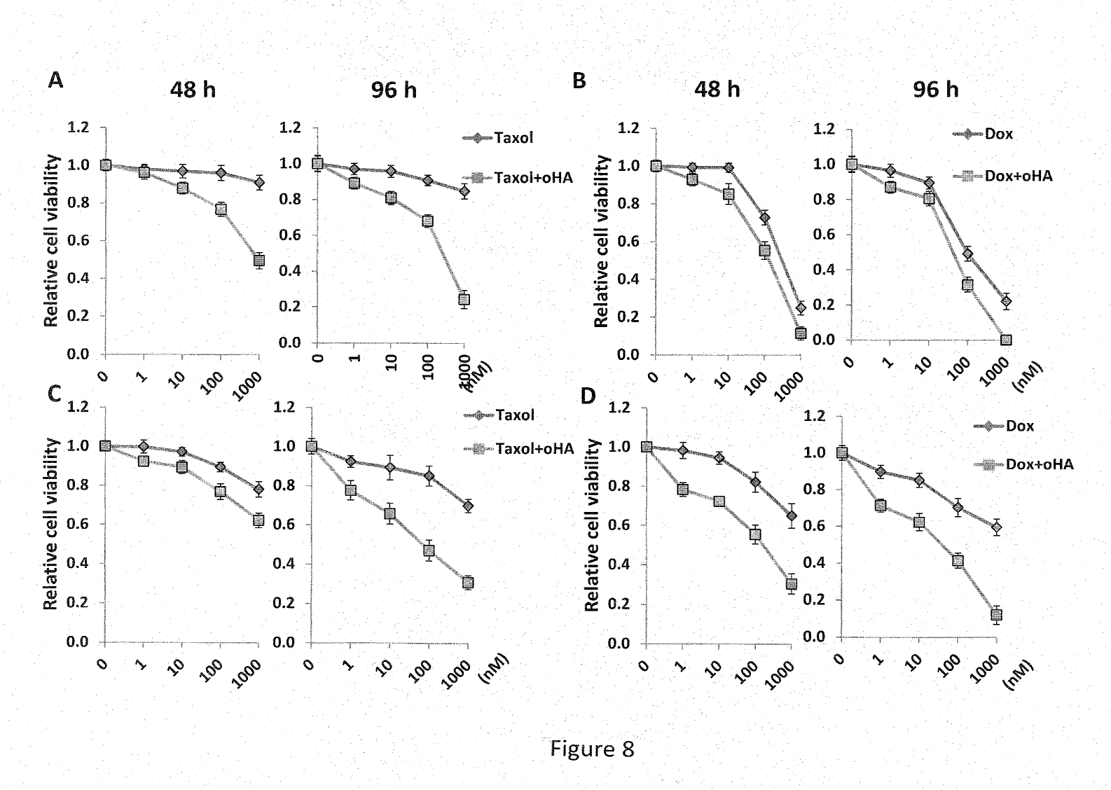

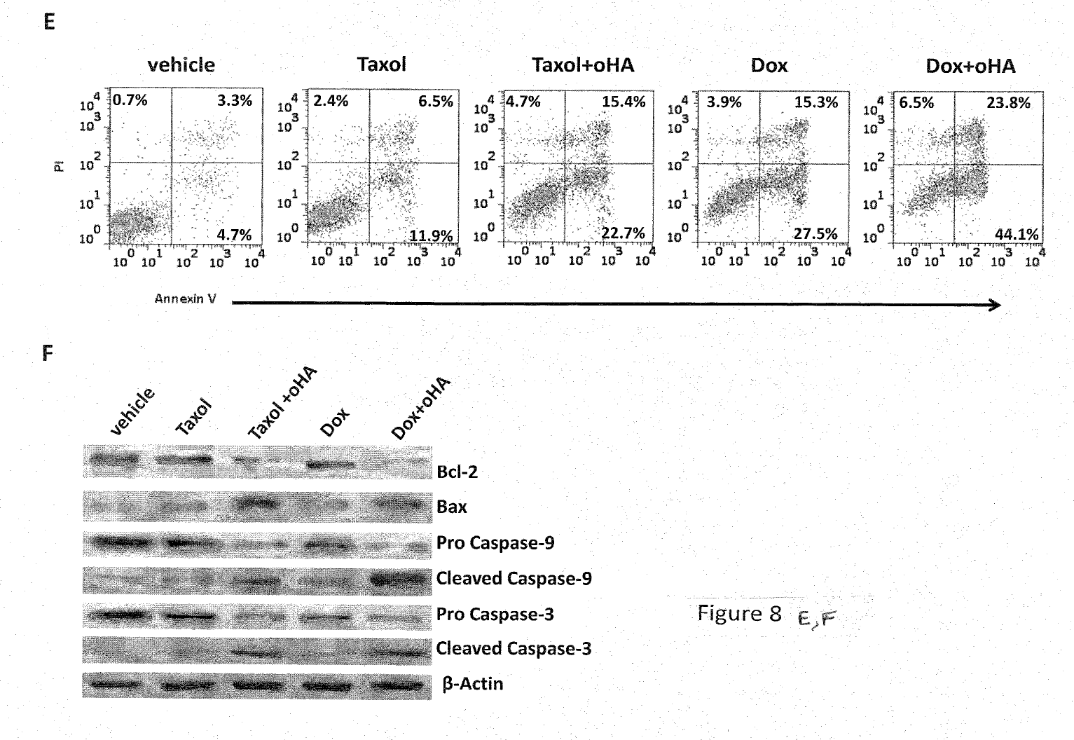

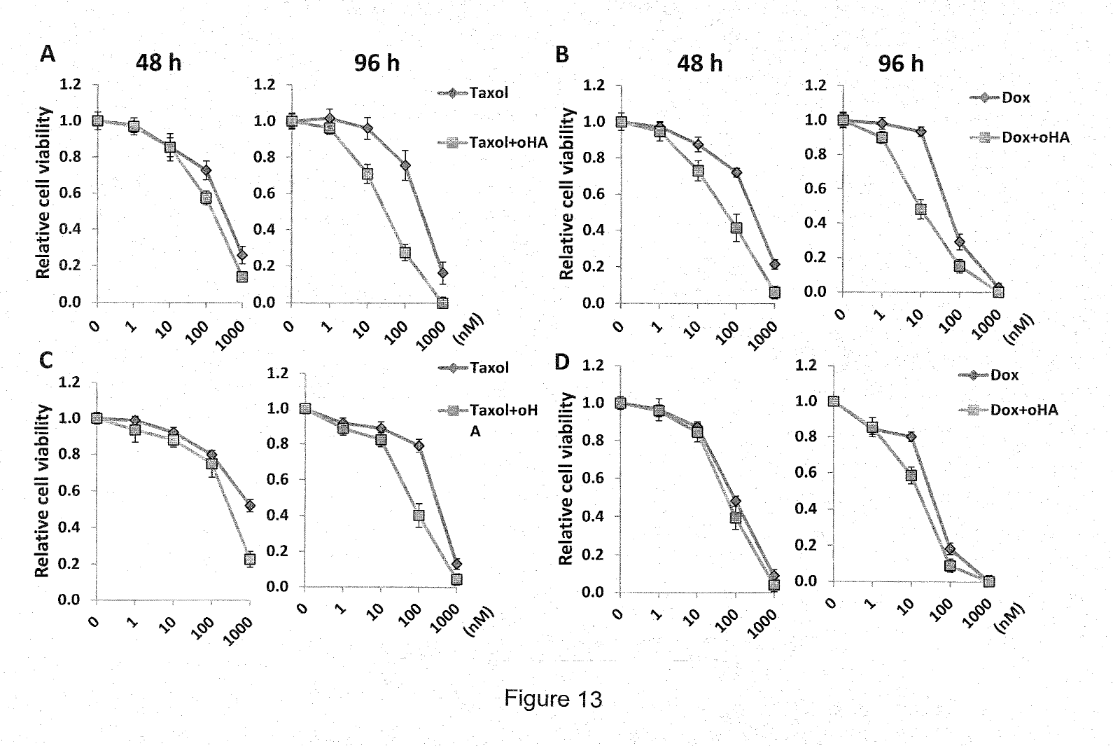

However, data obtained in examples herein showed that oHAs significantly enhanced PEL cytotoxicity induced by either doxorubicin or paclitaxel, with this effect being more pronounced for chemoresistant PEL cells (FIG. 8 panels A-D). FIG. 13 compare BC-1 cells (A,B) and BC-3 cells (C,D). In this experiment relative cell viability was determined in the presence of taxol or Dox, alone or with each of these drugs in the presence of oHA. oHAs was observed to have enhanced doxorubicin or paclitaxel induction of PEL apoptosis (FIG. 8 panel E). In parallel with the data obtained from the cells in FIG. 8 panel E, immunoblots were performed to identify apoptosis-associated protein expression as described in the examples, supra. Data are shown in FIG. 8 panels E and F for one of three independent experiments. These confirmed that oHAs reduced expression of the anti-apoptotic protein Bc1-2 (B-cell lymphoma 2), increased expression of the pro-apoptotic protein Bax and increased expression of the functional, pro-apoptotic cleaved proteins caspase-9 and caspase-3 while reducing the pro-forms of these proteins (FIG. 8 panel F). This latter observation is caused, in part, by a reduction of emmprin and BCRP expression with oHAs.

Collectively, these data support a role for hyaluronan-receptor interactions in the induction of PEL chemoresistance, and demonstrate that disruption of these interactions enhances chemotherapy-mediated apoptosis for PEL cells.

### Example 20

### oHAs Suppress Drug-Induced Expression of Emmprin and BCRP

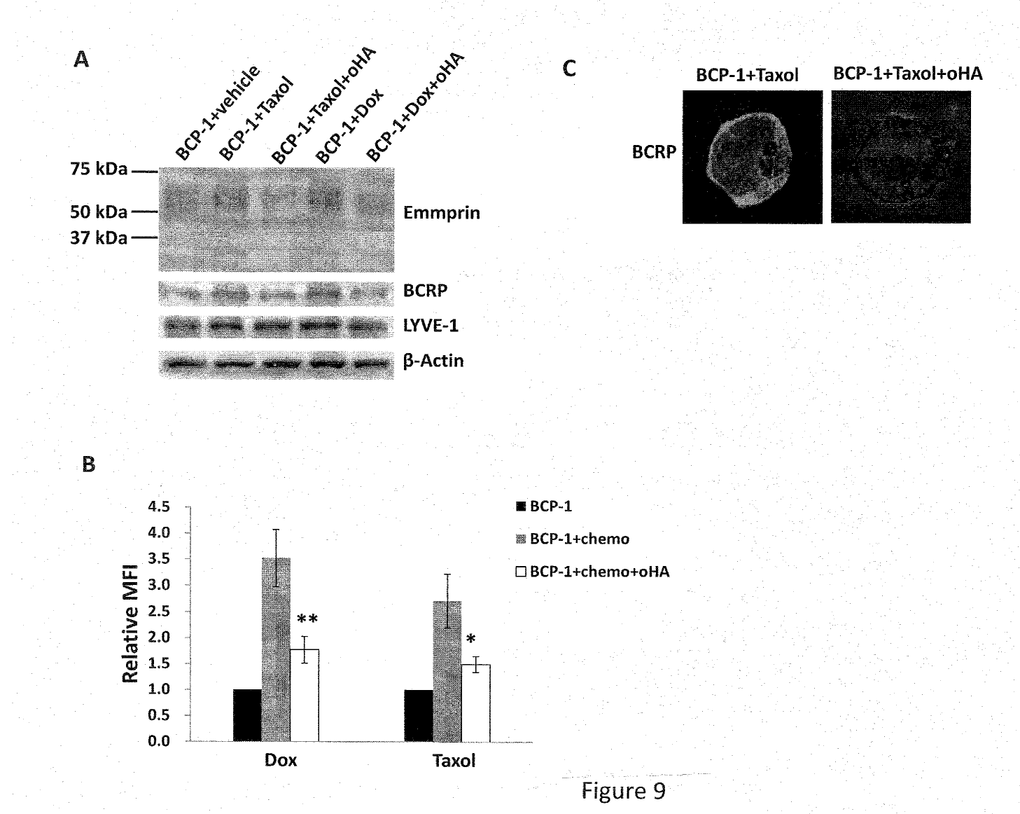

BCP-1 cells in this example were incubated with 100 nM Taxol or 100 nM Dox for 96 h in the presence or absence of 100 μg/ml oHA. Immunoblot analyses were used to detect total protein expression, including β-actin for internal controls. Data shown in FIG. 9 panel A represent one of three independent experiments. Flow cytometry analyses were used to quantify BCRP cell surface expression for similar conditions and mean fluorescence intensity (MFI), reflecting surface expression of BCRP for 10⁴ cells was determined for experimental groups relative to untreated BCP-1 control cells as shown in FIG. 9 panel B. Error bars represent the s.e.m. for three independent experiments, * indicates P less than 0.05; ** P less than 0.01. FIG. 9 panel C shows confocal IFAs of BCP-1 cells treated as in panel A, and imaged for identification and localization of BCRP expression as described in the examples, supra. Data shown represent one of three independent experiments.

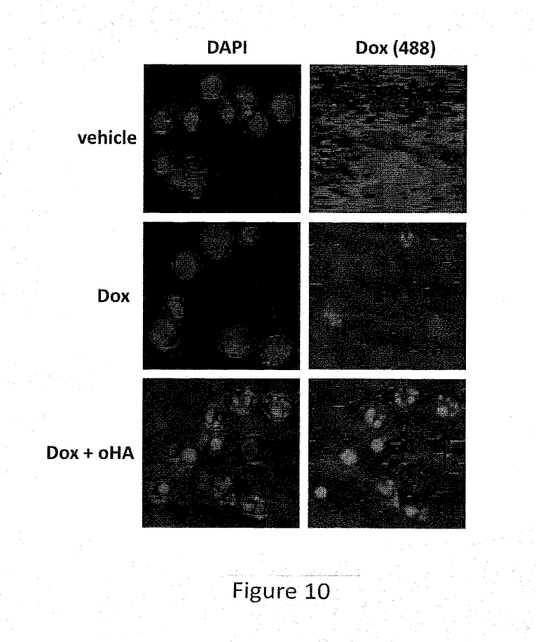

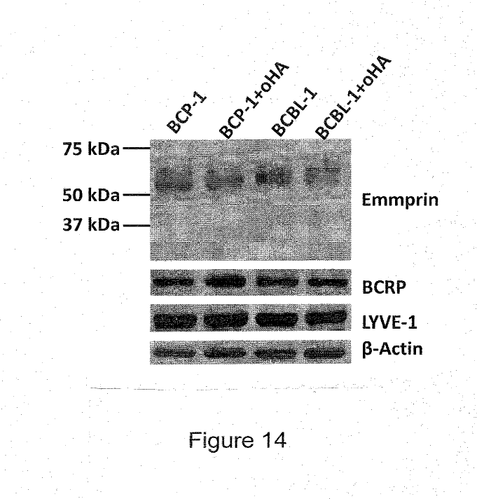

The immunoblots of FIG. 9 panel A show that oHAs suppressed doxorubicin- or paclitaxel-induced expression of emmprin and BCRP but not LYVE-1. However, oHAs alone had no significant impact on basal expression of emmprin, LYVE-1 or BCRP (FIG. 14, showing protein expression of BCP-1 and BCBL-1 cells cultured with oHA and in the absence of either chemotherapeutic drug). In addition, oHAs suppressed doxorubicin- or paclitaxel-induced cell surface expression of BCRP (FIG. 9 panels B and C). Laser excitation of intrinsic fluorescence for doxorubicin has been recently reported by Melloro H R, et al. Cancer Chemother Pharmacol 2011; 1179-1190, and the data in examples herein confirmed that intracellular accumulation of doxorubicin occurred in a significantly greater number of oHA-treated cells in these assays. Furthermore, intracellular accumulation of doxorubicin correlated with the degree of apoptosis for individual cells as determined by visualization of nuclear fragmentation (shown in FIG. 10, infra).

Collectively, these data support a role for hyaluronan-receptor interactions in the induction of PEL chemoresistance, and demonstrate that disruption of these interactions enhances chemotherapy-mediated apoptosis for PEL cells.

### Example 21

### oHA Potentiates Effect of Rapamycin as an Anti-Rumor Agent

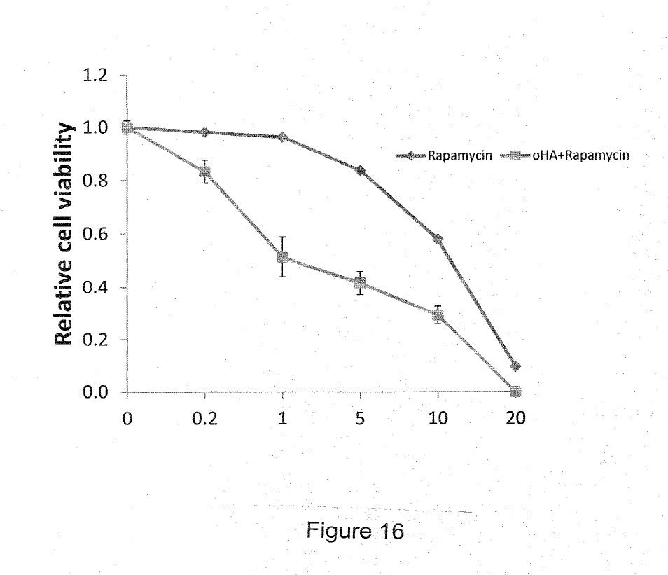

The potential effect of oHA in combination with antitumor agents is exemplified by analyses of rapamycin cell killing of BCBL-1 primary effusion lymphoma (PEL) cells in culture, as shown in FIG. 16. It is desirable for chemotherapeutic agents that tumor cell killing be achieved with the lowest possible concentration of the chemotherapeutic agent, to minimize side effects on the recipient of the agent. Accordingly, the twenty-fold increase in effectiveness resulting from using oHA in combination with rapamycin for causing cell death indicates that comparable anti-cancer effects are obtained at a 20-fold lower dose of the active anti-cancer agent. As seen in FIG. 16, which plots drug concentration in nm on the ordinate, a concentration of 1 nM of rapamycin, using this agent alone, resulted in almost no cell killing (survival greater than 0.95). In contrast, the combination of oHA and rapamycin resulted in cell death of about half the cells in the population, an extent of cell killing observed with rapamycin alone only at a much higher concentration of this drug, from about 10 to 20 nM. Thus oHA substantially potentiates rapamycin effectiveness.

Treatment of lymphoma patients such as those having PEL, has in the past involved rapamycin in some cases, but only limited success has been obtained. Clearly, combination therapy with oHA would greatly improve the rate of a successful outcome using the same standard dose regiment of rapamycin, and the combination might possibly even be equally or more effective than the current standard, at lower doses of rapamycin in combination with oHA.

### Example 21

### oHA Potentiate in vivo Anti-Cancer Effects of Rapamycin Killing of Lymphoma Cells

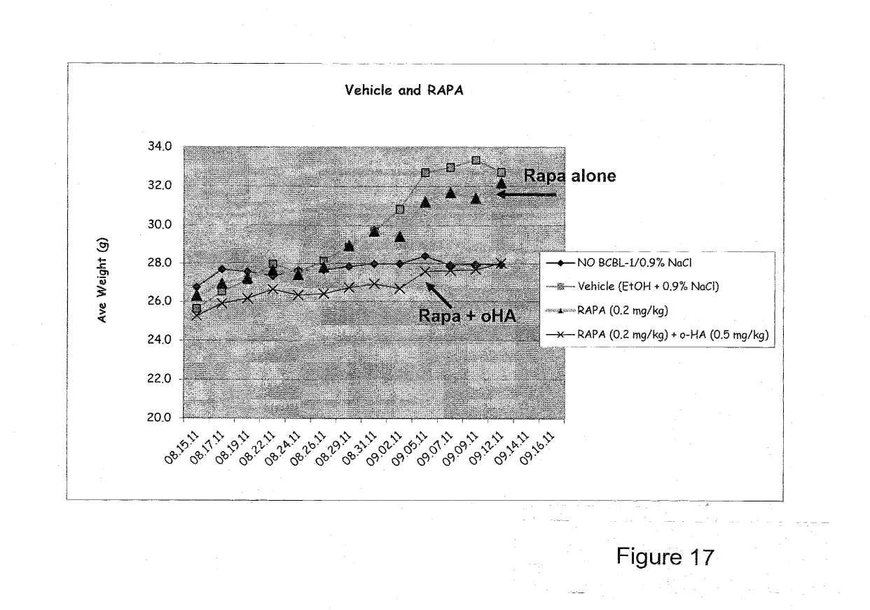

FIG. 17 is a line graph showing effect of oHA in combination with rapamycin on growth of tumors in BCBL-1-injected NOD/SCID mice. Mice were injected with 2×10⁷ BCBL-1 cells (a strain of PEL cells) and were weighed as a function of time every other day for one month, to assess tumor growth. During the course of the one-month analysis of the subjects in this animal model of lymphoma, the rapamycin alone did not significantly affect the increase in weight associated with lymphoma growth. In contrast, treatment with the combination of rapamycin and o-HA substantially reduced or even eliminated the weight gain associated with the progress of lymphoma in this mouse model system, as mouse weight was similar to that of control mice not injected with BCBL-1 cells (diamonds).

These data support therapeutic use of a combination of oHA with rapamycin to potentiate the effects of the treatment agent, and is expected to allow use of a lower dose or concentration of rapamycin or other anti-cancer agents than currently required, thus avoiding dose-dependent adverse effects while not sacrificing treatment efficacy.

### Example 22

### oHA Potentiates in vivo Anti-Cancer Effect of Doxorubicin Killing of Lymphoma Cells

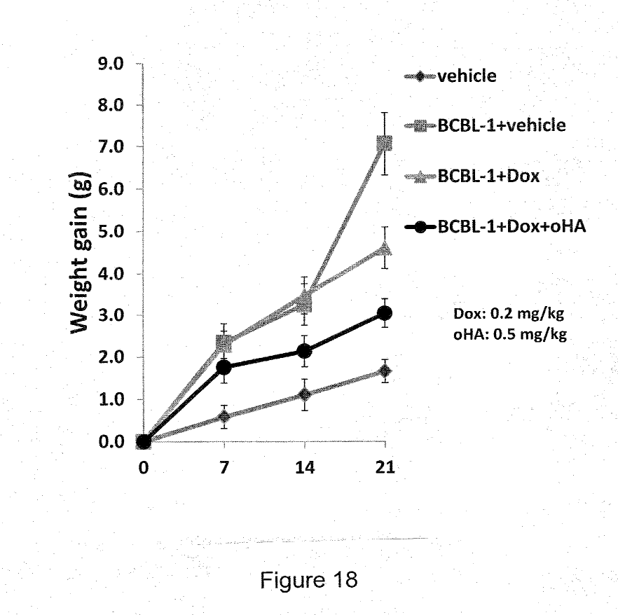

FIG. 18 is a line graph showing effect in vivo of oHA in combination with doxorubicin on growth of tumors and resulting increase in weight in BCBL-1-injected NOD/SCID mice. Mice were injected with 2×10⁷ BCBL-1 cells (a strain of PEL cells) and were weighed as a function of time every week for three weeks, to assess tumor growth. Weight was compared to control mice not receiving BCBL-1 cells.

Over the course of the three -week analysis of the subjects in the animal model of lymphoma, doxorubicin alone only slightly reduced the increase in weight associated with lymphoma growth in untreated mice. In contrast, treatment with the combination of doxorubicin and oHA substantially reduced the weight gain associated with the progress of lymphoma in this mouse model system. As shown, mouse weight gain was only about one gram more than seen with control mice that had not been injected with the BCBL-1 tumor cells (diamonds).

Treatment of lymphoma patients such as those having PEL, has in the past commonly involved doxorubicin, but only limited success has been obtained. The foregoing data support therapeutic use of a combination of oHA with doxorubicin to potentiate the effects of these chemotherapy agents, and/or to permit use of a lower dose or concentration of doxorubicin or other anti-cancer agents without lowering treatment effectiveness.

### Example 24

### Virus Gene Products Act to Upregulate Cell Receptors Involved in oHA Binding

A common feature of viral infection is expression of viral proteins that function to alter levels of expression of cell proteins. Viruses that cause cancer include KSV and EBV, and these viruses change expression of genes encoding cell receptors.

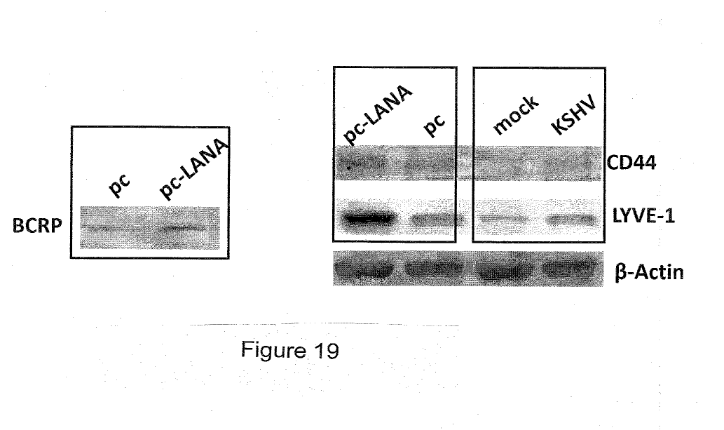

FIG. 19 shows western blot data illustrating upregulation of protein expression following primary human endothelial cell (EC) infection with KSHV, or EC transfection by the KSHV-encoded protein: LANA. EC extracts analyzed in the panel on the left were transformed with a vector encoding LANA (pc-LANA) or a control vector (pc), and expression of BCRP was analyzed and shown to be upregulated by LANA. EC extracts in the right panel show that LANA also upregulates expression of CD44 and LYVE-1, as does KHSV infection in comparison to uninfected EC (mock). Actin expression was used as a loading control and was not affected by any of these treatments. Thus, infection of cells with KSHV, or transformation with a KSHV gene product called LANA, induced an increase in expression of BCRP (a cell surface receptor associated with breast cancer), and of CD44 and LYVE-l. CD44 and LYVE-1 cell surface proteins are both known to bind hyaluronan, and these data suggest that these cell surface receptors are present in higher numbers in infected cells relative to uninfected cells. Expression of actin, a control housekeeping protein used to relative protein loading during gel electrophoresis, was not affected by any of these treatments.

Thus, these data show that oHA is more readily bound by transformed cells of a virus-associated lymphoma cell, or virus-infected precancerous cells, as receptors known to have affinity for hyaluronan are present in increased numbers on these cells. Most important, oHA functions to reverse resistance to drugs by virus-associated lymphoma cells through suppression of expression of proteins regulated by hyaluronan (like CD 147 and BCRP) as shown in Qin Z, et al. 2011; Leukemia 25: 1598-1605 which is hereby incorporated herein by reference in its entirety for all purposes, including references herein to observed color in an image or graph appearing in the corresponding image or graph of that published article.

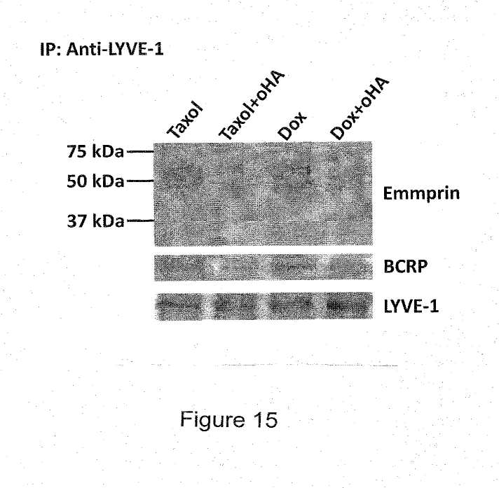

Other work has demonstrated that blocking hyaluronan interactions with CD44 disrupts emmprin- and CD44-drug efflux pump complexes on the cell surface (Slomiany M G, et al. Clin Cancer Res 2009; 15(24): 7593-7601), and it was observed herein that oHAs reduced co-precipitation of LYVE-1 with either emmprin or BCRP (FIG. 15). It is envisioned that additional experiments resolve whether oHAs reduce emmprin and BCRP expression in PEL cells treated with chemotherapeutic agents.

### Example 25

### oHA Inhibits Expression of Activated pAkt and p-mTOR

BCBL-1 cell were cultured in this example in the presence of doxorubicin, or doxorubicin and oHA and western blot data were collected to determine the levels various proteins, including activated Akt (p-Akt) and activated mTOR (p-mTOR). These proteins represent important signaling pathways in tumorigenesis.

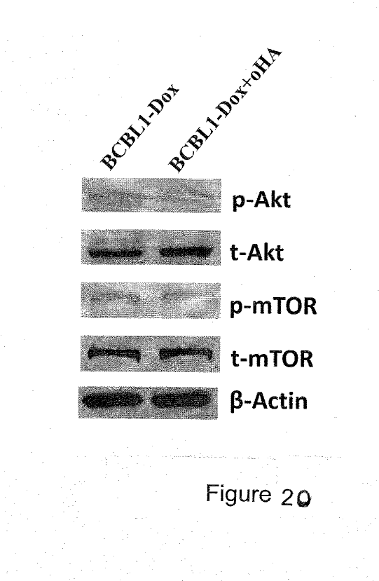

FIG. 20 shows the observed blots, with β-actin analyzed as a control. No differences were observed in total expression of total Akt or mTOR, but oHA substantially inhibited expression of the activated forms, both p-Akt and p-mTOR.

Cytotoxic chemotherapeutic agents represent the current standard of care for PEL, but these agents may aggravate toxicities associated with antiretroviral agents administered to HIV- infected patients and have not improved the poor prognosis for patients with these tumors (Petre C E, et al. J Virol 2007; 81(4): 1912-1922; Simonelli C, et al. J Clin Oncol 2003; 21(21): 3948-3954; Boulanger E, et al. J Clin Oncol 2005; 23(19): 4372-4380; Chen Y B, et al. Oncologist 2007; 12(5): 569-576). Sensitization of PEL to existing chemotherapies permits dose reduction of cytotoxic agents to minimize associated toxicities, as well as augmentation of chemotherapy-mediated PEL apoptosis to improve clinical outcomes. Data from a single report suggest that mutation of p53 leads to doxorubicin resistance for PEL cells (Petre C E, et al. J Virol 2007; 81(4): 1912-1922). A second report found that the KSHV-encoded LANA2 modulates microtubule dynamics through direct binding to polymerized microtubules, thereby interfering with microtubule stabilization by paclitaxel and increasing PEL resistance to this drug (Munoz-Fontela C, et al. J Virol 2008; 82(3): 1518-1525). However, neither of these mechanisms of resistance can be easily targeted for therapeutic purposes, supporting the need for identification of alternative mechanisms for PEL resistance, specifically those involving potential targets at the cell surface.

Emmprin, through interactions with hyaluronan receptors (Slomiany M G, et al. Cancer Res 2009; 69(4): 1293-1301; Misra S, et al. J Biol Chem 2003; 278(28): 25285-25288) and membrane-bound transporters (Slomiany M G, et al. Cancer Res 2009; 69(4): 1293-1301; Slomiany M G, et al. Clin Cancer Res 2009; 15(24): 7593-7601; Wang W J, et al. Chemotherapy 2008; 54(4): 291-301), facilitates tumor cell chemoresistance. In addition, disruption of hyaluronan interactions with its cognate receptors interferes with emmprin- mediated drug resistance (Misra S, et al. J Biol Chem 2003; 278(28): 25285-25288), in part through disruption of protein complexes containing emmprin (Slomiany M G, et al. Cancer Res 2009; 69(4): 1293-1301; Slomiany M G, et al. Clin Cancer Res 2009; 15(24): 7593-7601). Examples herein sought to determine whether emmprin, the hyaluronan receptor LYVE-1 and the ABC-family multidrug transporter BCRP regulate PEL resistance to chemotherapy. This approach was initially supported by observing a direct correlation between PEL resistance to chemotherapeutic agents and expression of emmprin, LYVE-1, and BCRP, as well as hyaluronan secretion (FIG. 1), and data supporting interactions for these proteins on the PEL cell surface (FIG. 2).

Data in examples herein are believed to be the first that establish roles for either emmprin or LYVE-1 in the regulation of BCRP expression, and previous data demonstrated decreased expression of BCRP by glioma cells after oHA treatment (Gilg, A. G., et al. Clin Cancer Res. 14:1804-1813, 2008). The examples herein are consistent with data indicating that increased emmprin expression stimulates hyaluronan—CD44 interactions (Marieb E A, et al. Cancer Res 2004; 64(4): 1229-1232; Misra S, et al. J Biol Chem 2003; 278(28): 25285-25288), which in turn increase expression of another ABC family transporter, P-glycoprotein (Misra S, et al. J Biol Chem 2005; 280(21): 20310-20315; Bourguignon L Y, et al. J Biol Chem 2009; 284(5): 2657-2671). However, we have found that P-glycoprotein is not expressed to an appreciable extent by PEL cells.

The BCRP promoter contains a CAAT box and Sp1-binding sites (Doyle L A, et al. Oncogene 2003; 22(47): 7340-7358). Emmprin and LYVE-1 regulate signal transduction pathways (Misra. S, et al. J Biol Chem 2003; 278(28): 25285-25288; Venkatesan B, et al. J Mol Cell Cardiol 2010; 49(4): 655-663; Tang Y, et al. Mol Cancer Res 2006; 4(6): 371-377; Huang Z, et al. Biochem Biophys Res Commun 2008; 374(3):517-521; Saban M R, et al. Blood 2004; 104(10): 3228-3230) that are known to regulate transcriptional activation through cooperative mechanisms involving CAAT box and Sp1 binding (Benjamin J T, et al. J Immunol 2010; 185(8): 4896-4903; Stein B, et al. Mol Cell Biol 1993; 13(7): 3964-3974).

KSHV- encoded LANA has been shown to induce expression of emmprin (Qin Z, et al. Cancer Res 2010; 70(10): 3884-3889). Sp1 also induces transcriptional activation of emmprin (Kong L M, et al. Cancer Sci 2010; 101(6): 1463-1470), and LANA interacts directly with Sp1 to promote Sp1-mediated transcriptional activation of telomerase (Verma S C, et al. J Virol 2004; 78(19): 10348-10359). Further, KSHV infection of primary human fibroblasts isolated from the oral cavity results in enhanced secretion of KS-promoting cytokines and instrinsic invasiveness through a VEGF-dependent mechanism and these effects are induced through Sp1- and Egr2-dependent transcriptional activation of emmprin (Dai, L et al. 2011; Cancer Lett epub ahead of print December 17). Examples herein indicate that neither emmprin nor LYVE-1 regulate expression of one another, and it is envisioned that these two proteins are functionally interdependent by virtue of their interactions. KSHV has thus been shown to induce endothelial cell expression of CD147 (emmprin), and of CD44, and LYVE-1. Further, presence of oHA dissociates the emmprin reduces emmprin expression. As emmprin is needed for full KSHV induction of endothelial cell invasion and emmprin induces endothelial cell invasion through activation of ERK and other signal transduction components, then it is clear that oHA can reduce or even eliminate effects of KSHV infection and its association with cancer.

It is here envisioned that oHA will be a useful therapeutic regimen in a variety of different virus-associated cancers, including those mediated by KSHV, other strains of HSV, human papillomavirus infection associated with cervical carcinoma (Yaqin et al. M 2007; Scan J Infect Dis 39: 441-448) and tongue and tonsil cancers (Lindquist D et al. 2012; Anticancer Res 32:153-162), hepatitis B virus X (Lara-Pezzi E et al. 2001; Hapatology 33: 1270-1281), HIV and cervical intraepithelial neoplasia (Darai E et al. 2000; Gynecolog Oncol 76: 56-62) and other retroviruses (Boulware D et al. 2011; J Infect Diseasese 203:1637-1646), co-infection with HIV and hepatitis virus C (Nunes D 2010; Am J Gasteroenterology 105: 1346-1353). In each of these virus-associated cancers, it is envisioned herein that oHA co-administration with an anticancer agent would result in sensitization of cancer cells to an anticancer chemotherapeutic agent and even a physical agent such as X-rays, resulting in an improved prognosis of remediation of the cancer, and potential decreased dosage of the anticancer agent, providing the patient with greater comfort, improved outcome, and fewer side effects, better quality of life, and decreased medical costs.

Examples herein show that either oHA treatment or direct LYVE-1 silencing suppresses BCRP expression and enhances PEL cytotoxicity in the presence of chemotherapeutic agents. The data support the possibility that hyaluronan interactions with LYVE-1 on the PEL cell surface facilitate PEL chemoresistance through upregulation of BCRP expression. Although its function as a receptor for hyaluronan is well characterized (Jackson D G. Immunol Rev 2009; 230(1): 216-231), this is the first report to our knowledge implicating LYVE-1 in downstream regulation of a membrane transport protein important for chemotherapeutic resistance, and the first report detailing a mechanism for LYVE-1 regulation of KSHV-associated cancer pathogenesis despite the fact that LYVE-1 expression has been reported within Kaposi's sarcoma lesions (Pyakurel P, et al. Int J Cancer 2006; 119(6): 1262-1267).

Published studies implicated interactions between emmprin and the hyaluronan receptor CD44 in the induction of cancer cell chemo-resistance (Misra S, et al. J Biol Chem 2003; 278(28): 25285-25288; Toole B P, et al. Drug Resist (pdat 2008; 11(3): 110-121). In addition, oHAs disrupt emmprin—CD44 interactions (Slomiany M G, et al. Cancer Res 2009; 69(4): 1293-1301) as well as CD44-mediated intracellular signal transduction and cell pathogenesis relevant to cancer progression (Slomiany M G, et al. Clin Cancer Res 2009; 15(24): 7593-7601; Marieb E A, et al. Cancer Res 2004; 64(4): 1229-1232; Misra S, et al. J Biol Chem 2003; 278(28): 25285-25288; Misra S, et al. J Biol Chem 2005; 280(21): 20310-20315; Cordo Russo R I, et al. Int J Cancer 2008; 122(5): 1012-1018; Ghatak S, et al. J Biol Chem 2002; 277(41): 38013-38020; Ghatak S, et al. J Biol Chem 2005; 280(10): 8875-8883). However, further data obtained using methods herein showed that both total and membrane expression of CD44 were negligible for the PEL cell lines used in these examples, in agreement with published results (Boshoff C, et al. Blood 1998; 91(5): 1671-1679). Results of data from examples herein are interpreted to include the possibility that oHAs enhance PEL cytotoxicity through disruption of hyaluronan interactions with a receptor other than or in addition to either CD44 or LYVE-1 (Zhou B, et al. J Biol Chem 2000; 275(48): 37733-37741; Hamilton S R, et al. J Biol Chem 2007; 282(22): 16667-16680), or through other mechanisms.

Examples herein show that direct targeting of emmprin or LYVE-1 using RNAi, and treatment with oHAs, enhance chemotherapy- induced apoptosis for PEL cells. As none of these interventions induced apoptosis in the absence of cytotoxic agents, and as emmprin-enhanced viability for PEL cells was reduced by targeting BCRP, data in examples herein indicate that targeting emmprin or LYVE-1 augments chemotherapy-induced PEL apoptosis through inhibition of BCRP expression and drug efflux. This is supported by our observation that chemotherapeutic agents increase emmprin expression by PEL cells in a manner previously observed for other cancer cell types (Li Q Q, et al. Cancer Sci 2007; 98(11): 1767-1774). Since emmprin stimulates hyaluronan synthesis (Marieb E A, et al. Cancer Res 2004; 64(4): 1229-1232), and the effect of emmprin on drug resistance is most likely mediated by hyaluronan-receptor interactions (Misra S, et al. J Biol Chem 2003; 278(28): 25285-25288), it is likely that chemotherapeutic agents also stimulate hyaluronan—LYVE-1 signaling and that oHAs act by interfering with this signaling. In addition, we observed an increase in the number of PEL cells exhibiting intracellular accumulation of doxorubicin in the presence of oHAs, further supporting the conclusion that oHAs inhibit drug efflux by effects on transporter expression (Slomiany M G, et al. Clin Cancer Res 2009; 15(24): 7593-7601; Slomianyn M G, et al. Cancer Res 2009; 69(12): 4992-4998; Gilg, A. G., et al. Clin Cancer Res. 14:1804-1813, 2008; Misra S, et al. J Biol Chem 2005; 280(21): 20310-20315) Emmprin and LYVE-1 also activate signal transduction pathways, including mitogen-activated protein kinase, phosphatidylinositol 3-kinase/Akt and nuclear factor-kB (Misra S, et al. J Biol Chem 2003; 278(28): 25285-25288; Venkatesan B, et al. J Mol Cell Cardiol 2010; 49(4): 655-663; Tang Y, et al. Mol Cancer Res 2006; 4(6): 371-377; Huang Z, et al. Biochem Biophys Res Commun 2008; 374(3): 517-521; Saban M R, et al. Blood 2004; 104(10): 3228-3230), that regulate apoptosis (Keshet Y, et al. Methods Mol Biol; 661: 3-38; Stiles B L. Adv Drug Daily Rev 2009; 61(14): 1276-1282; Kawauchi K, et al. Anticancer Agents Med Chem 2009; 9(5): 550-559; Shen H M, et al. Apoptosis 2009; 14(4): 348-363).

Constitutive activation of these pathways plays a pivotal role in anti- apoptotic signaling and PEL cell survival (Ford P W, et al. J Gen Virol 2006; 87(Pt 5): 1139-1144; Tomlinson C C, et al. J Virol 2004; 78(4): 1918-1927; Cannon M L, et al. Oncogene 2004; 23(2): 514-523; Sin S H, et al. Blood 2007; 109(5): 2165-2173), and inhibition of these pathways induces PEL apoptosis (Sin S H, et al. Blood 2007; 109(5): 2165-2173; Uddin S, et al. Clin Cancer Res 2005; 11(8): 3102-3108; Takahashi-Makise N, et al. Int J Cancer 2009; 125(6): 1464-1472; Keller S A, et al. Blood 2000; 96(7): 2537-2542). It is possible that inhibition of emmprin or LYVE-1 also induces PEL apoptosis through interference with signal transduction.

Data in examples herein show that emmprin, LYVE-1 and BCRP colocalize and interact on the PEL cell surface. Recent reports suggest that emmprin interacts with CD44 (Slomiany M G, et al. Cancer Res 2009; 69(4): 1293-1301) and P-glycoprotein (Slomiany M G, et al. Clin Cancer Res 2009; 15(24): 7593-7601; Wang W J, et al. Chemotherapy 2008; 54(4): 291-301), thereby facilitating drug efflux and resistance to chemotherapy. It is likely that emmprin and CD44 interact with several plasma membrane proteins within the context of lipid rafts rather than through direct binding to one another (Ghatak S, et al. J Biol Chem 2005; 280(10): 8875-8883; Bourguignon, L. Y., et al. J Biol Chem 2004; 279: 26991-27007; Tang, W., et al. J Biol Chem 2004; 279: 11112-11118), and whether emmprin, LYVE-1 and BCRP interact in this manner on the PEL cell surface is currently under investigation.

Moreover, oHAs inhibit drug efflux activity and sensitize tumor cells to chemotherapy through disruption of hyaluronan—CD44—drug transporter interactions and internalization of both CD44 and drug transporters (Slomiany M G, et al. Clin Cancer Res 2009; 15(24): 7593-7601; Slomiany M G, et al. Cancer Res 2009; 69(12): 4992-4998; Misra S, et al. J Biol Chem 2005; 280(21): 20310-20315) in addition to their effects on transporter expression. Data in examples herein show that emmprin or LYVE-1 targeting with RNAi, or treatment with oHAs, reduced total BCRP expression in PEL cells. Using confocal immunofluorescence assays, we also observed a reduction of PEL membrane localization of BCRP with these interventions, but without coincident increases in cytoplasmic BCRP expression; however, these findings do not categorically exclude the possibility that BCRP is internalized and degraded as a result of emmprin or LYVE-1 targeting or oHA treatment. In addition, although oHAs reduced co-immunoprecipitation of emmprin, LYVE-1 and

BCRP, it is possible that the observed reduction in BCRP protein expression with oHA treatment contributes to reduced quantitative interactions between these proteins at the cell surface. Additional experiments should clarify which of these mechanisms for emmprin/LYVE-1 regulation of BCRP play a key role in protecting PEL cells from apoptosis and cytotoxicity induced by chemotherapeutic agents.

The foregoing observations and data support the potential utility of targeting one or more of these intermediates as a therapeutic approach for PEL and other KSHV-associated and other virus-associated diseases, particularly viruses such as herpes strains, retroviruses such as HIV, and human papilloma virus, hepatitis viruses Band C, and for virus-associated cancers such as cervical, tongue, tonsillar, Kaposi's sarcoma, and PEL.

The invention in various embodiments now having been fully described, additional embodiments are exemplified by the following Examples and claims, which are not intended to be construed as further limiting. The contents of all cited references are hereby incorporated by reference herein.

What is claimed is:

1. A method for treating a resistant cancer, tumor, precancerous cell, or virus-infected cell resistant to a chemotherapeutic agent, the cell characterized by having at least one marker induced by, upregulated by or associated with chronic virus exposure and promoting drug resistant cell and/or tumor growth in an associated tissue, wherein the method comprises the step of administering a competitor of hyaluronan interactions in an amount effective to overcome invasiveness, drug resistance or metastasis characteristics of the cell or tumor, or to induce cell death for a virus-infected cell or tumor.

2. The treatment method according to claim 1, wherein the competitor of hyaluronan interactions is applied in association with a drug to which the cell is resistant absent the competitor, thereby sensitizing the cell to the drug so that tumor growth is controlled while administering a low dose of drug.

3. The treatment method according to claim 1, wherein the competitor of hyaluronan interactions is selected from at least one of the group of competitors consisting of: i) small hyaluronan oligomers of 2-20 disaccharides length to compete with hyaluronan for binding sites; ii) a composition that modulates expression or binding capacity of a cell surface hyaluronan binding protein; iii) a composition that modulates expression or activity of hyaluronan; and iv) a composition that modulates expression or activity of a protein active in a downstream hyaluronan-utilizing pathway.

4. The treatment method according to claim 1, wherein the at least one marker is selected from the group of: emmprin (CD147), CD44, and lymphatic vessel hyaluronan receptor-1 (LYVE-1).

5. The treatment method according to claim 1, wherein the at least one marker mediates a signal transduction pathway, thereby activating blood vessel growth and tissue invasion.

6. The treatment method according to claim 1, wherein the virus-infected cells, precancerous cells, and/or cancerous cells express a drug transporter protein.

7. The treatment method according to claim 1, wherein the drug transporter protein includes breast cancer resistance protein ABCG2 (BCRP).

8. The treatment method according to claim 1, wherein the virus-infected cells, precancerous cells, and/or cancerous cells are cells of primary effusion lymphoma (PEL) or Kaposi's sarcoma (KS) lesion.

9. The treatment method according to claim 1, wherein the marker comprises a phenotype associated with exposure to at least one virus selected from: an Epstein-Barr virus (EBV), a Kaposi's sarcoma-associated herpesvirus (KSHV), a Hepatitis B virus (HBV), an Hepatitis C virus (HCV), a Human Papilloma virus (HPV), a polyomavirus, and a Human Immunodeficiency virus (HIV).


SEQUENCE LISTING (8 sequences)

SEQ ID NO: 1 - DNA, 18 bp, Artificial Sequence
  Hyalruonan synthase 1 sense
caaggcgctcggagattc

SEQ ID NO: 2 - DNA, 20 bp, Artificial Sequence
  Hyalruonan synthase 1 antisense
gaccgctgatgcaggataca

SEQ ID NO: 3 - DNA, 18 bp, Artificial Sequence
  Hyalruonan synthase 2 sense
catcatccaaagcctgtt

SEQ ID NO: 4 - DNA, 19 bp, Artificial Sequence
  Hyalruonan synthase 2 antisense
tcttctgagttcccatcta

SEQ ID NO: 5 - DNA, 18 bp, Artificial Sequence
  Hyalruonan synthase 3 sense
tggctcaaccagcaaacc

SEQ ID NO: 6 - DNA, 20 bp, Artificial Sequence
  Hyalruonan synthase 3 antisense
cagcaggaagaggagaatgt

SEQ ID NO: 7 - DNA, 19 bp, Artificial Sequence
  beta-actin sense
ggaaatcgtgcgtgacatt

SEQ ID NO: 8 - DNA, 21 bp, Artificial Sequence
  beta-actin anti sense
gactcgtcatactcctgcttg